SEQUENCE LISTING (29 sequences)

SEQ ID NO: 1 - RNA, 93 nt, Artificial Sequence
  RNA aptamer for CEA
gggagagcggaagcgugcugggcuagaauaauaauaagaaaaccaguacuuucgugucccgggagggugcauaacccagaggucgauggaucc

SEQ ID NO: 2 - RNA, 93 nt, Artificial Sequence
  RNA aptamer for CEA
gggagagcggaagcgugcugggcuagaauaauaauaagaaaaccaguacuuucgugugccgggcggguccauaacccagaggucgauggaucc

SEQ ID NO: 3 - RNA, 93 nt, Artificial Sequence
  RNA aptamer for CEA
gggagagcggaagcgugcugggcuagaauaauaauaagaaaaccaguacuuucgugucccgggagguuccauaacccagaggucgauggaucc

SEQ ID NO: 4 - RNA, 93 nt, Artificial Sequence
  RNA aptamer for CEA
gggagagcggaagcgugcugggcuagaauaauaauaagaaaaccaguacuuucgugucccgggaggagccauaacccagaggucgauggaucc

SEQ ID NO: 5 - RNA, 93 nt, Artificial Sequence
  RNA aptamer for CEA
gggagagcggaagcgugcugggcuagaauaauaauaagaaaaccaguacuuucgugucccgggaggacacauaacccagaggucgauggaucc

SEQ ID NO: 6 - RNA, 93 nt, Artificial Sequence
  RNA aptamer for CEA
gggagagcggaagcgugcugggcuagaauaauaauaagaaaaccagaacuuucgugucccgggagguuucauaacccagaggucgauggaucc

SEQ ID NO: 7 - RNA, 93 nt, Artificial Sequence
  RNA aptamer for CEA
gggagagcggaagcgugcugggcuagaauaauaauaagaaaaccaguacuuucgcgucccaggaggguccauaacccagaggucgauggaucc

SEQ ID NO: 8 - RNA, 94 nt, Artificial Sequence
  RNA aptamer for CEA
gggagagcggaagcgugcugggcuagaauaauaauaagaaaaccaguacuuucguguccccgggaggauucauaacccagaggucgauggaucc

SEQ ID NO: 9 - RNA, 95 nt, Artificial Sequence
  RNA aptamer for CEA
gggagagcggaagcgugcugggcuagaauaauaauaagaaaaaccaguacuuucgugucccgggagagguacauaacccagaggucgauggaucc

SEQ ID NO: 10 - RNA, 93 nt, Artificial Sequence
  RNA aptamer for CEA
gggagagcggaagcgugcugggcucgaauaauaauaacgaaaaccaguacuuucgugucccgggaggaccauaacccagaggucgauggaucc

SEQ ID NO: 11 - RNA, 92 nt, Artificial Sequence
  RNA aptamer for CEA
gggagagcggaagcgugcugggcucgaauaauaauaagaaaaccaguacuuucgugucccgggagggccauaacccagaggucgauggaucc

SEQ ID NO: 12 - RNA, 92 nt, Artificial Sequence
  RNA aptamer for CEA
gggagagcggaagcgugcugggcuagaauaauaauaagaaaaccaguacuuucgugucccgggagggccauaacccagaggucgauggaucc

SEQ ID NO: 13 - RNA, 49 nt, Artificial Sequence
  RNA aptamer for CEA
gcggaagcgugcugggcuagaauaauaauaagaaaaccaguacuuucgu

SEQ ID NO: 14 - RNA, 35 nt, Artificial Sequence
  RNA aptamer for CEA
gugcugggcuagaauaauaauaagaaaaccaguac

SEQ ID NO: 15 - RNA, 49 nt, Artificial Sequence
  Mutant RNA aptamer for CEA
gcggaagcgugcugggcuagggcggcggcgggaaaaccaguacuuucgu

SEQ ID NO: 16 - RNA, 35 nt, Artificial Sequence
  Mutant RNA aptamer for CEA
gugcugggcuagggcggcggcgggaaaaccaguac

SEQ ID NO: 17 - RNA, 23 nt, Artificial Sequence
  Loop region
ggcuagaauaauaauaagaaaac

SEQ ID NO: 18 - RNA, 95 nt, Artificial Sequence
  RNA aptamer for CEA
gggagagcggaagcgugcugggcumgaauaauaauaanraaaaccagwacuuucgygusccnrgrvrgdnncauaacccagaggucgauggaucc

SEQ ID NO: 19 - DNA, 32 bp, Artificial Sequence
  Full-CEA-5′-primer
cccaagcttagaccatggagtctccctcggcc

SEQ ID NO: 20 - DNA, 44 bp, Artificial Sequence
  Full-CEA-3′-primer
gctctagactatatcagagcaaccccaaccagcactccaatcat

SEQ ID NO: 21 - DNA, 26 bp, Artificial Sequence
  N domain 5′-primer
cgaattcaagctcactattgaatcca

SEQ ID NO: 22 - DNA, 30 bp, Artificial Sequence
  B3 domain 3′-primer
cccaagcttctaagatgcagagactgtgat

SEQ ID NO: 23 - DNA, 27 bp, Artificial Sequence
  A1 domain 5′-primer
cgaattcaagccctccatctccagcaa

SEQ ID NO: 24 - DNA, 32 bp, Artificial Sequence
  N domain 3′-primer
cccaagcttctacagctccgggtatacccgga

SEQ ID NO: 25 - DNA, 30 bp, Artificial Sequence
  A1 domain 3′-primer
cccaagcttctacgggccatagaggacatt

SEQ ID NO: 26 - DNA, 49 bp, Artificial Sequence
  5′-primer for linking site between N and A1 domains
tggccagttccgggtataccgggagctgtccaagccctccatctccagc

SEQ ID NO: 27 - DNA, 49 bp, Artificial Sequence
  3′-primer for linking site between N and A1 domains
gctggagatggagggcttggacagctcccggtatacccggaactggcca

SEQ ID NO: 28 - DNA, 41 bp, Artificial Sequence
  5′-primer for RNA library
ggtaatacgactcactatagggagagcggaagcgtgctggg

SEQ ID NO: 29 - DNA, 28 bp, Artificial Sequence
  3′-primer for RNA library
ggggggatccatcgacctctgggttatg

(19) United States
(12) Patent Application Publication
(10) Pub. No.: US 2011/0184042 A1
(43) Pub. Date: Jul. 28, 2011

(54) RNA Aptamer Specifically Binding to Carcinoembryonic Antigen and Use thereof

(75) Inventors: SEONG-WOOK LEE, SEOUL (KR); YOUNG-JU LEE, SEOUL (KR); JIN-SOOK JEONG, BUSAN (KR)

(73) Assignees: POSTECH ACADEMY-INDUSTRY FOUNDATION, POHANG-CITY (KR); POSCO, POHANG-SHI (KR)

(21) Appl. No.: 12/725,529

(22) Filed: Mar. 17, 2010

Related U.S. Application Data
(60) Provisional application No. 61/160,912, filed on Mar. 17, 2009.

(51) Int. Cl. C07H 21/02; A61K 31/7105; C12Q 1/68

(52) U.S. Cl. 514/44 A; 536/23.1; 435/6.14

(57) ABSTRACT

Provided are RNA aptamer specifically binding to cancer metastasis-inducing domain of CEA (Carcinoembryonic antigen), a composition for prevention and/or inhibition and/or diagnosis of cancer metastasis containing the same as an active ingredient, and a method of prevention and/or inhibition and/or diagnosis of cancer metastasis using the same.

### CROSS-REFERENCE TO RELATED APPLICATION

This application claims priority to and the benefit of U.S. Provisional Application No. 61/169,912 filed in the United States Patent and Trademark Office on Mar. 17, 2009, the entire contents of which are incorporated herein by reference.

### BACKGROUND OF THE INVENTION

(a) Field of the Invention This disclosure relates to RNA aptamer specifically binding to a cancer metastasis-inducing domain of CEA (Carcinoembryonic antigen), a composition for prevention and/or inhibition and/or diagnosis of metastasis containing the RNA aptamer as an active ingredient, and a method of prevention and/or inhibition and/or diagnosis of metastasis using the RNA aptamer.

(b) Description of the Related Art

CEA (Carcinoembryonic antigen: CEACAM5, NCBI accession number: NP_{—}004354), which is 180 KDa glycophosphatidylinositol (GPI)-anchored membrane glycoprotein, is heavily glycosylated. The CEA is consisted of N domain consisting of 107 amino acids at N-terminal, and 6 domains (A1, B1, A2, B2, A3, B3) repeated similarly to Ig(Immunoglobulin), each consisting of 178 amino acids, and the C-terminal is consisted of glycosylphosphatidylinositol membrane anchor. CEA has been studied as a cancer marker, and expressed in 70% of lung cancer and 50% of breast cancer, and colon cancer, stomach cancer and pancreatic cancer. Due to such characteristic, it is widely used clinically to diagnose cancer. CEA expression is known to be related to cell adhesion, inhibition of apoptosis and promotion of metastasis to liver. The level of CEA in blood is mainly used as a basis for determining prognosis after colon cancer surgery.

N domain of CEA is known to increase cell aggregation through the process of inducing interaction between CEA-positive (CEA-expressing) cells, thereby playing an important role to induce metastasis. In particular, 5 amino acids ‘PELPK’ existing between N domain and A1 domain of CEA is known to be responsible for binding to kupffer cell that is associated with metastasis. It has been known that the ‘PELPK’ is recognized by 80 kDa cell surface receptor on kupffer cell and greatly activate the next receptors by signal transduction, whereby CEA-expressing cells are brought into liver to induce metastasis.

In addition, since CEA is a Ca²⁺ independent intercellular adhesion molecule between homotypic cells, a possibility that a metastasis of CEA-expressing cells may occur by permeation through cell membrane to develop into cancer is suggested. As the result of analyzing CEA amino acid sequence of patients who have a large amount of CEA in blood stream but do not have metastasis to liver, among the patients with CEA-induced diseases, it is confirmed that PELPK region of CEA is mutated, This result suggests a possibility that mutation in PELPK may inhibit binding affinity of CEA to the receptor on the kupffer cell of liver, thereby inhibiting metastasis to liver, suggesting that a ligand to a cancer specific marker may be used as a strong means for cancer diagnosis and development of an effective anticancer therapeutic agent.

### SUMMARY OF THE INVENTION

The present inventors developed a novel RNA molecule specifically binding to a linkage region between N domain and A1 domain of a cancer-specific marker CEA (Carcinoembryonic antigen), thereby being useful for inhibition and diagnosis of metastasis, to complete the invention.

One embodiment of the present invention provides an RNA molecule consisting essentially of a specific region within a polynucleotide of SEQ ID NO: 18. The RNA molecule may comprise a nucleotide sequence selected from the group consisting of SEQ ID NO: 1 to 14.

Another embodiment provides an RNA aptamer specifically binding to a linkage region between N domain and A1 domain of CEA, and essentially comprising a specific region within a polynucleotide of SEQ ID NO: 18. The RNA aptamer may comprise a nucleotide sequence selected from the group consisting of SEQ ID NO: 1 to 14.

Another embodiment provides a composition for inhibition and/or prevention of cancer metastasis, containing an RNA aptamer that specifically binds to a linkage region between N domain and A1 domain of CEA, and essentially comprises a specific region within a polynucleotide of SEQ ID NO: 18, and a method of inhibition and/or prevention of cancer metastasis comprising administering said RNA aptamer to a patient in need of inhibition and/or prevention of cancer metastasis. The RNA aptamer may comprise a nucleotide sequence selected from the group consisting of SEQ ID NO: 1 to 14.

Another embodiment provides a composition for diagnosis of cancer metastasis, containing an RNA aptamer that specifically binds to a linkage region between N domain and A1 domain of CEA, and essentially comprises a specific region within a polynucleotide of SEQ ID NO: 18, and a method of diagnosis of cancer metastasis using said RNA aptamer. The RNA aptamer may comprise a nucleotide sequence selected from the group consisting of SEQ ID NO: 1 to 14.

### DETAILED DESCRIPTION OF THE EMBODIMENTS

The present invention relates to an RNA molecule specifically binding to a linkage region between N domain and A1 domain of a cancer-specific marker CEA (Carcinoembryonic antigen), use of said RNA molecule as an RNA aptamer for CEA, and inhibition/prevention and diagnosis technologies of metastasis using said RNA aptamer.

RNA aptamer specific to a target protein with high affinity can be separated from an RNA library comprising random polynucleotides through in vitro selection technology using SELEX method. The aptamer can be synthesized chemically, easily resynthesized, and is inexpensive, and can be used for highly specific diagnosis. In particular, the RNA aptamer has several characteristics that it is capable of stabilized structure formation and reversible denaturation, it does not cause immunoreaction that is a main limitation of antibody, and the conditions for binding between the aptamer and a target is controllable. Thus, RNA aptamer can function as a molecule replacing antibody, drug, and the like that can bind to a clinically related target molecule, because it can be easily synthesized, has high affinity and specificity, and does not have immunogenicity, which has been a problem of the use of antibody.

An embodiment provides a novel RNA molecule comprising continuous 35 or more bases essentially comprising the nucleotide sequence from 15^{th} to 50^{th} positions of SEQ ID NO: 18.

<SEQ ID NO: 18>
GGGAGAGCGG AAGCGUGCUG GGCUMGAAUA AUAAUAANRA
AAACCAGWAC UUUCGYGUSC CNRGRVRGDN NCAUAACCCA
GAGGUCGAUG GAUCC

wherein,

M is A or C;

N is A or U or G or C or absent (i.e., deleted);

R is G or A;

W is A or U;

Y is U or C;

S is G or C;

V is A or G or C (i.e., not U);

D is A or G or U (i.e., not C); and

each N positioned at 38 and 62 is independently G or C or absent (deleted), and each N positioned at 70 and 71 is independently A or U or G or C.

The minimum number of bases of the nucleotide sequence from 15^{th} to 50^{th} positions of SEQ ID NO: 18 is 35, since N located in the nucleotide sequence from 15^{th} to 50^{th} positions of SEQ ID NO: 18 may be absent (i.e., ‘deleted N’); as shown in Example 7, an RNA molecule comprising the 35 bases is capable of specifically binding to CEA; and thus, the nucleotide sequence from 15^{th} to 50^{th} positions of SEQ ID NO: 18 is determined as a minimum functional unit in the present invention.

According to one concrete embodiment, the RNA molecule may comprise a nucleotide sequence selected from the group consisting of SEQ ID NO: 1 to 14. Particularly, the RNA molecule may comprise continuous 35 or more bases essentially comprising the nucleotide sequence from 9^{th} to 43^{rd} positions of SEQ ID NO: 13. The RNA molecule comprising a nucleotide sequence of SEQ ID NO: 13 or 14 has excellent specificity and affinity to CEA, and thus is very useful as RNA aptamer for CEA.

Another embodiment provides a use of the RNA molecule as CEA specific RNA aptamer. Thus, a CEA specific RNA aptamer comprising continuous 35 or more bases essentially comprising the nucleotide sequence from 15^{th} to 50^{th} positions of the SEQ ID NO: 18 is provided. The RNA aptamer specifically binds to a linkage region between N domain and A1 domain of CEA (see Examples 5 to 7). The linkage region between N domain and A1 domain of CEA may comprise 5 amino acids, ‘PELPK’.

The RNA aptamer may comprise a polynucleotide selected from the group consisting of SEQ ID NO: 1 to 14. Particularly, the RNA molecule may comprise continuous 35 or more bases comprising a 9 to 43 polynucleotide of SEQ ID NO: 13. The RNA molecule comprising a polynucleotide of SEQ ID NO: 13 or 14 has excellent specificity and affinity to CEA, and thus is very useful as RNA aptamer to CEA.

For allowing resistance to RNase, the RNA aptamer may be modified in several manners. For example, pyrimidine bases C(cytosine) and U(uracil) wherein 2′ hydroxyl group is substituted with a fluoro group may be used, and/or a cholesterol molecule may be attached to 5′ end of the RNA aptamer and inverted dT(idT) may be attached to 3′ end.

As explained, the linkage region (for example, PELPK) between N domain and A1 domain of CEA is recognized by the receptor on other organ's cells, in particular by the receptor on kupffer cell, and plays an important function for metastasis of CEA-expressing cell to other organs, in particular liver. Therefore, the RNA aptamer binding to the region may be useful for inhibition or diagnosis of metastasis of CEA-expressing cell to other organs, particularly liver.

Accordingly, another embodiment provides a composition for inhibition and/or prevention of cancer metastasis containing the RNA aptamer as an active ingredient, and a method of inhibition and/or prevention of cancer metastasis comprising administering the RNA aptamer to a patient in need of inhibition and/or prevention of cnacer metastasis.

The RNA aptamer may comprise a nucleotide sequence selected from the group consisting of SEQ ID NO: 1 to 14, and as explained, it may be modified for resistance to RNase, as described above.

The cancer on which the RNA aptamer has the effect of metastasis inhibition/prevention may be any CEA-related cancer, and for example, it may be selected from the group consisting of colon cancer, stomach cancer, pancreatic cancer, lung cancer, etc., but not limited thereto. The metastasis, on which the RNA aptamer has the inhibition/prevention effect, may be one to any organ capable of recognizing CEA, for example, liver. Preferably, the RNA aptamer may be useful for inhibition of metastasis of colon cancer to liver.

The RNA aptamer may be administered to any mammals, preferably rodents, livestock, human, etc., and more preferably human. A route of administration is not specifically limited and any administration route may be used. For example, it may be administered orally, intravenously, intramuscularly, subcutaneously, and preferably it may be administered to the affected part by intravenous injection. The RNA aptamer may be administered together with commonly used additives such as pharmaceutically acceptable carrier, excipient, and/or diluents.

A dose of RNA aptamer may be within the range not showing liver toxicity, and it may be commonly administered in the amount of 100 ug/kg (body weight) to 2000 ug/kg (body weight) per a day, preferably 500 ug/kg (body weight) to 1000 ug/kg (body weight) per a day, and more preferably about 800 ug/kg (body weight) per a day, but the dose may be appropriately adjusted depending on the age, body weight and severity of disease of patient.

Another embodiment provides a composition for diagnosis of cancer metastasis containing the RNA aptamer, and a method of diagnosis of cancer metastasis using the RNA aptamer. The RNA aptamer may comprise the nucleotide sequence selected from the group consisting of SEQ ID NO: 1 to 14, and as described, it may be modified for resistance to RNase.

The cancer on which the RNA aptamer has metastasis inhibition effect may be any CEA-related cancer, for example, it may be selected from the group consisting of colon cancer, stomach cancer, pancreatic cancer, lung cancer, etc., but not limited thereto. The metastasis, on which the RNA aptamer has inhibition effect, may be one to any organ capable of recognizing CEA, for example, liver. Preferably, the RNA aptamer may be useful for inhibition of metastasis of colon cancer to liver.

According to one concrete embodiment, the method of diagnosis of metastasis comprises

treating a sample or a patient with the RNA aptamer, and

detecting binding of the RNA aptamer and CEA (Carcinoembryonic antigen), more particularly a linkage region between N domain and A1 domain,

wherein it is determined that metastasis occurs when the binding is detected.

The binding of the RNA aptamer and CEA may be detected by any conventional means, and for example, RNA aptamer may be conventionally labeled and detected. The label may be any conventional fluorescences, radioisotopes, etc., and the fluorescences or radioisotopes may be detected by common detection means (for example, Radio immune guided surgery (RIGS), Radioimmunodetection (RAID), etc.) to determine binding of the RNA aptamer and CEA.

The RNA aptamer may be useful for in vivo diagnosis as well as ex vivo diagnosis, and it may be directly applied to a living body as well as be applied to a tissue or cell separated from mammals, preferably human, to diagnose cancer metastasis.

Another embodiment provides a method of molecular imaging using the RNA aptamer to trace and visualize cancer cell.

More specifically, the molecular imaging method may comprise

applying the above RNA aptamer that is labeled with a fluorescence or radioisotope to a living body or an isolated tissue or cell; and

detecting the fluorescence or radioisotope.

By labeling the RNA aptamer with conventional fluorescence or radioisotope, applying it to a living body or an isolated tissue or cell of mammals including human (for example, by intravenous administration, etc) and detecting the fluorescence or radioisotope by a conventional method, CEA-expressing cancer cell can be traced and the location and distribution thereof can be visualized (for example, see FIGS. 15 to 17). The CEA-expressing cancer cell may be a cancer cell of cancer selected from the group consisting of colon cancer, stomach cancer, pancreatic cancer, lung cancer, etc.

### BRIEF DESCRIPTION OF DRAWINGS

FIG. 1 is an overview of SELEX procedure for CEA.

FIG. 2A shows nucleotide sequences of selected RNAs, and 2B-2D show secondary structures thereof.

FIG. 3A shows nucleotide sequences of optimized RNA, and 3B-3F show secondary structures thereof.

FIG. 4 is a sensogram of optimized RNA aptamer.

FIG. 5 shows 5′-cholesterol-modified RNA aptamer for CEA.

FIGS. 6A-6F show results of homotypic cell aggregation inhibition assay, indicating Ca²⁺-independent inhibition of homotypic cell aggregation by RNA aptamer for CEA.

FIG. 7 is a graph showing the result of in vitro ECM adhesion assay.

FIG. 8 is a graph showing the result of in vitro ECM adhesion inhibition assay by YJ-1 and Mutant aptamer treatment.

FIG. 9 is a graph showing the result of Collagen-Based cancer cell Invasion inhibition Assay.

FIGS. 10A and 10B show the result of CEA-induced metastasis protection assay in animal model.

FIG. 11 shows the result of CEA-induced metastasis prevention assay in animal model.

FIG. 12 is a graph showing the result of liver toxicity test.

FIG. 13 is a graph showing the result of In vitro cell migration assay.

FIG. 14 is a graph showing the result of Anoikis inducing Assay.

FIGS. 15 to 17 show staining images of CEA-positive (CEA-expressing) cancer cell when being treated with CEA Aptamer.

### EXAMPLE

The present invention is further explained in more detail with reference to the following examples. These examples, however, should not be interpreted as limiting the scope of the present invention in any manner.

### Example 1

### Preparation of CEA Protein

1.1: Construction of protein

CEA protein was constructed by preparing the following primers based on Full CEACAM5 (CEACAM5 NCBI accession number: NP_{—}004354) and cloning:

Full-CEA-5′-primer:
(SEQ ID NO: 19)
5′-CCCAAGCTTAGACCATGGAGTCTCCCTCGGCC-3′
Full-CEA-3′-primer:
(SEQ ID NO: 20)
5′-GCTCTAGACTATATCAGAGCAACCCCAACCAGCACTCCAATCAT-3′

Vector DNA was purified through Midiprep procedure using Midiprep kit (Promega, PureYield™ Plasmid Midiprep System).

To purify protein (N.CEA) consisting of N domain to B3 domain, N domain-specific primer was used as 5′-primer, and B3 domain-specific primer was used as 3′-primer.

N domain 5′-primer:
(SEQ ID NO: 21)
5′-CGAATTCAAGCTCACTATTGAATCCA-3′
B3 domain 3′-primer:
(SEQ ID NO: 22)
5′-CCCAAGCTTCTAAGATGCAGAGACTGTGAT-3′

To purify protein (A.CEA) consisting of A1 domain to B3 domain, A1 domain-specific primer was used as 5′-primer, and B3 domain-specific primer was used as 3′-primer:

A1 domain 5′-primer:
(SEQ ID NO: 23)
5′-CGAATTCAAGCCCTCCATCTCCAGCAA-3′
B3 domain 3′-primer:
(SEQ ID NO: 22)
5′-CCCAAGCTTCTAAGATGCAGAGACTGTGAT-3′

To purify N domain protein, N domain-specific primer was used as 5′-primer, and N domain-specific primer was used as 3-'primer.

N domain 5′-primer:
(SEQ ID NO: 21)
5′-CGAATTCAAGCTCACTATTGAATCCA-3′
N domain 3′-primer:
(SEQ ID NO: 24)
5′- CCCAAGCTTCTACAGCTCCGGGTATACCCGGA -3′

To purify A1 domain protein, A1 domain-specific primer was used as 5′-primer, and A1 domain-specific primer was used as 3′-primer:

A1 domain 5′-primer:
(SEQ ID NO: 23)
5′- CGAATTCAAGCCCTCCATCTCCAGCAA -3′
A1 domain 3′-primer:
(SEQ ID NO: 25)
5′- CCCAAGCTTCTACGGGCCATAGAGGACATT-3′

To purify protein consisting of N domain and A1 domain, N domain-specific primer was used as 5′-primer, and A1 domain-specific primer was used as 3′-primer:

N domain 5′-primer:
(SEQ ID NO: 21)
5′-CGAATTCAAGCTCACTATTGAATCCA -3′
A1 domain 3′-primer:
(SEQ ID NO: 25)
5′-CCCAAGCTTCTACGGGCCATAGAGGACATT -3′

To construct mutant protein wherein 5 amino acids PELPK of the linkage region between N domain and A1 domain are converted to RELSK, a two-step procedure was conducted (See Tuerk, C., Gold, L. (1990) Systematic evolution of ligands by exponential enrichment:RNA ligands to bacteriophage T4 DNA polymerase. Science, 249, 505-510). As 5′-primer, 5′-TGGCCAGTTCCGGGTATA CCGGGAGCTGTCCAAGCCCTCCATCTCCAGC-3′(SEQ ID NO: 26) with 2 mutated nucleic acids was used, and as 3′-primer, 5′-GCTGGAGATGGAGGGCTTGGACAGCTCCCGGTATACCCGGAACTGGCCA-3′(SEQ ID NO: 27) with 2 mutated nucleic acids was used. After conducting PCR, DNA was eluted and it was used as a template to conduct PCR under the following conditions.
Repeat (95° C. 30 seconds, 58° C. 30 seconds, 72° C. 1 minute 30 seconds) 30 times

All of the above structures were cloned into pET28a(+) vector (Novagen) using EcoR I and Hind III restriction enzyme (Roche Applied Science), and then, transformed into BL21 Escherichia coli (Invitrogen) by heat shock. Base sequence was identified by sequencing analysis.

1.2: Extraction of Protein

A 5 ml LB medium (tryptone 10 g/liter NaCl 10 g/liter yeast extract 5 g/liter (BD biosciences)) was inoculated with each protein stock prepared in Example 1.1, and grown at 37° C. for 16 hours to 18 hours. And then, a 500 ml LB medium was inoculated with the above 5 ml, and incubated at 37° C. until OD value reaches 0.6 to 0.8.

Protein extraction conditions included temperature, culture time, and IPTG (Isopropyl β-D-1-thiogalactopyranoside) concentration as described in TABLE 1. Cell lysis and sonication were conducted, and protein was eluted with controlling imidazole concentration (3-4 eluted proteins were obtained with elution buffer of imidazole concentration of 50 mM, 100 mM, and 250 mM, at each concentration), and concentrated and quantified by Bradford analysis.

TABLE 1
protein extraction condition

| | | | IPTG |
|---|---|---|---|
| | Temperature | Culture time | concentration |
| NCEA | 30° C. | 7 hrs | 2.5 mM |
| ACEA | 30° C. | 7 hrs | 2 mM |
| N only | 30° C. | 6 hrs | 1 mM |
| A1 only | 30° C. | 7 hrs | 0.5 mM |
| N + A1 domain | 37° C. | 7 hrs | 1 mM |
| Mut. N + A1 domain | 30° C. | 6 hrs | 1 mM |

### Example 2

### Construction of DNA Library

To construct a RNA library required for conducting SELEX procedure, according to a commonly known method, using a 76mer single oligonucleotide randomly including 40 bases as a template, a DNA library was constructed through PCR with 5′-primer(GGTAATACGACTCACTATAGGGAGAGCGGAAGCGTGCTGGG, SEQ ID NO: 28) and 3′-primer(GGGGGGATCCATCGACCTCTGGGTTATG, SEQ ID NO: 29). The 5′-primer includes T7 RNA region for synthesizing RNA.

0.25 μM 5′-primer, 0.25 μM 3′-primer, 10×PCR buffer (Promega), and 100 μM dNTP mixture (Roche Applied Science) were mixed, and 2.5 unit Taq polymerase (Promega) was added at initial 95° C., 5 minutes. And, as PCR cycles, 10 cycles of 95° C. 30 seconds, 55° C. 30 seconds, and 72° C. 1 minutes were repeated, and then, finally, 72° C. 8 minutes 30 seconds, to construct various DNA libraries.

### Example 3

### Construction of RNA Library

Using the DNA library with various base sequences constructed though PCR in Example 2 as a template, a RNA library was constructed through in vitro transcription. At this time, in order to prepare RNA resistant to RNase, by transcription of a template synthesized in vitro using 2′-deoxy-2′-fluoro CTP and UTP (Epicentre Technologies), normal GTP and ATP, and T7 RNA polymerase, RNA with each 2 position of pyrimidine nucleotide modified to fluoro group was produced (See Gold, L., Polisky, B., Uhlenbeck, O., Yarus, M. (1995) Diversity of oligonucleotide functions. Annu. Rev. Biochem. 64, 763-797).

The DNA library, 10× transcription buffer, 50 mM DTT, 5 mM ATP, 5 mM GTP, 5 mM 2′-F-CTP, 5 mM 2′-F-UTP, T7 RNA polymerase (Epicentre Technologies), DEPC-H₂O(DiethylenePyrocarbonate-H₂O) were used to adjust reaction volume to 20λ, and they were reacted at 37° C. for 6 hours. The reactant was treated with 1 MBU DNaseI (Epicentre Technologies) at 37° C. for 15 minutes to remove DNA used as template. An RNA library was eluted using Sephadex G25 column (sigma). RNA obtained through selection procedure was eluted from 7M urea-6% polyacrylamide gel.

### Example 4

### Selection of N+A1 Domain Specific RNA Aptamer

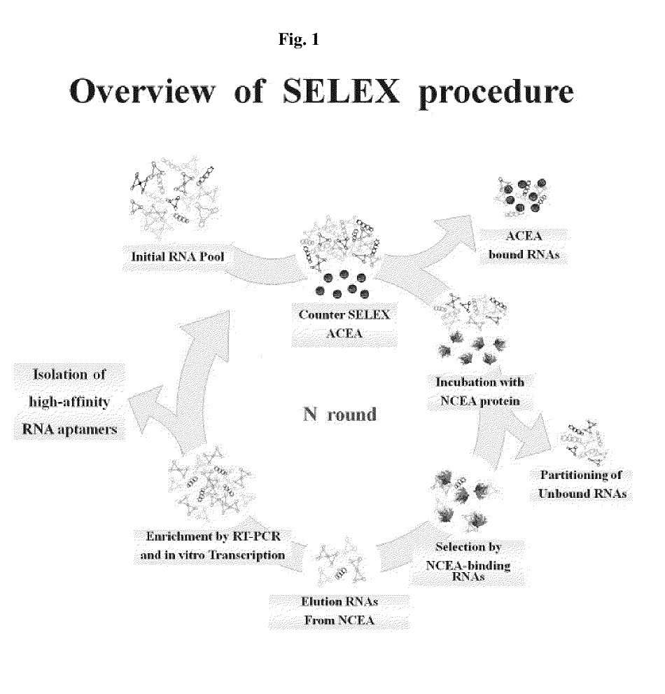

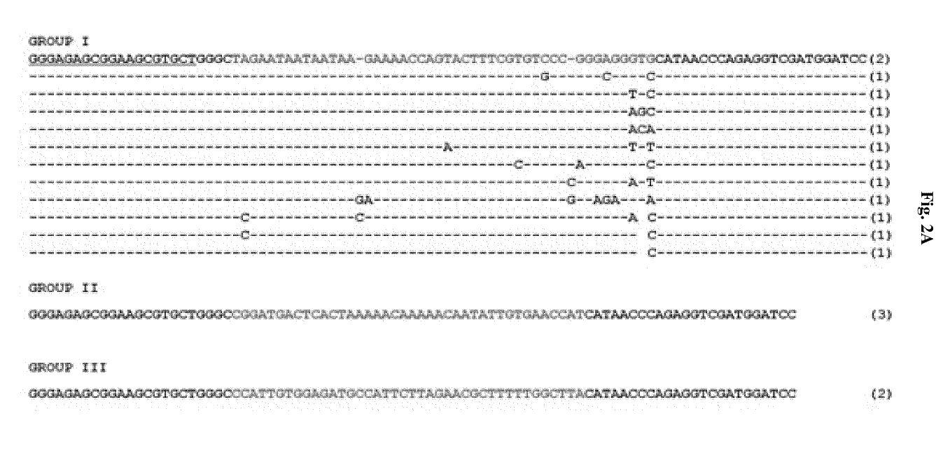

To detect RNA aptamers specifically binding to a specific CEA domain related to metastasis, which is used as a metastasis inhibitor according to the present invention, a counter selection method of removing ACEA-bound RNAs and then detecting NCEA-binding RNAs was used. FIG. 1 is a schematic drawing of SELEX procedure to CEA.

First, a preclearing step of removing ACEA-bound RNAs was conducted, and then, RNAs capable of specifically binding to NCEA was detected, thereby selecting RNA aptamers that can specifically bind to a specific domain (N+A1 domain) of CEA, which is used for a metastasis inhibitor of the present invention.

The RNA library constructed in Example 3 and ACEA constructed in Example 1 were reacted at room temperature for 20 minutes. And, Ni-NTA agarose beads (QIAGEN) were rapidly spinned and washed with binding buffer (30 mM Tris-HCl (PH 7.5), 150 mM NaCl, 1.5 mM MgCl₂, 2 mM DTT, 1% BSA), and then, reacted with the above reactant at room temperature for 20 minutes. After the reaction, only supernatant was taken and reacted with NCEA at room temperature for 20 minutes. Ni-NTA agarose beads (QIAGEN) washed by rapid spinning were taped together and reacted. Ni-NTA agarose beads (QIAGEN) and RNA and protein complex was washed with binding buffer (30 mM Tris-HCl (PH 7.5), 150 mM NaCl, 1.5 mM MgCl₂, 2 mM DTT, 1% BSA) 5 times repeatedly. And then, it was dissolved in TE buffer (10 mM Tris-Cl, pH 7.5, 1 mM EDTA (Sigma)), and NCEA-binding RNAs were eluted by phenol extraction and concentrated by ethanol precipitation.

SELEX 1st round was conducted as explained, and from 2nd round, NCEA-binding RNAs were amplified by the following method and used in the next cycle.

Into RNAs obtained through each cycle of SELEX, 500 nM of 3′-primer (5′-GGGGGGATCCATCGACCTCTGGGTTATG-3, SEQ ID NO: 29) was introduced, and denatured at 65° C. for 5 minutes, and then, left at room temperature for 10 minutes to bind RNA with the primer. 1 mM dNTP, 5×RT buffer (promega), and 25U AMV RTase(promega) were added and reacted at 37° C. for 30 minutes, and then, heated at 95° C. for 5 minutes, and cooled at 4° C. to inactivate reverse transcriptase. Synthesized cDNA was amplified by PCR. DNA obtained by reverse transcription-PCR was identified by 3% agarose gel, and RNA was synthesized again through in vitro transcription by the same method as constructing RNA library and used in the next selection process.

SELEX was conducted total 17 rounds. 1^{st} to 5^{th} rounds were conducted with the mole ratio of NCEA:ACEA:RNA of 1:2:2, and 6^{th} to 15^{th} rounds were conducted with the mole ratio of NCEA:ACEA:RNA of 1:10:2, thereby providing reliability to the counter selection procedure for removing RNAs nonspecifically binding to ACEA. 16^{th} to 17^{th} rounds were conducted with the mole ratio of NCEA:ACEA:RNA of 1:10:1 to finally remove RNAs capable of binding to ACEA.

TABLE 2
SELEX Condition

| Round | RNA | | Protein (ACEA) | Protein (NCEA) |
|---|---|---|---|---|
| 1st | 5 | μg (150 pmole) | 8 μg (138 pmole) | 5 μg (70 pmole) |
| 2^{nd} | 5 | μg (150 pmole) | 8 μg (138 pmole) | 5 μg (70 pmole) |
| 3rd | 5 | μg (150 pmole) | 8 μg (138 pmole) | 5 μg (70 pmole) |
| 4th | 5 | μg (150 pmole) | 8 μg (138 pmole) | 5 μg (70 pmole) |
| 5th | 5 | μg (150 pmole) | 8 μg (138 pmole) | 5 μg (70 pmole) |
| 6th | 1 | μg (30 pmole) | 8 μg (138 pmole) | 1 μg (14 pmole) |
| 7th | 1 | μg (30 pmole) | 8 μg (138 pmole) | 1 μg (14 pmole) |
| 8th | 1 | μg (30 pmole) | 8 μg (138 pmole) | 1 μg (14 pmole) |
| 9th | 1 | μg (30 pmole) | 8 μg (138 pmole) | 1 μg (14 pmole) |
| 10th | 1 | μg (30 pmole) | 8 μg (138 pmole) | 1 μg (14 pmole) |
| 11th | 1 | μg (30 pmole) | 8 μg (138 pmole) | 1 μg (14 pmole) |
| 12th | 1 | μg (30 pmole) | 8 μg (138 pmole) | 1 μg (14 pmole) |
| 13th | 1 | μg (30 pmole) | 8 μg (138 pmole) | 1 μg (14 pmole) |
| 14th | 1 | μg (30 pmole) | 8 μg (138 pmole) | 1 μg (14 pmole) |
| 15th | 1 | μg (30 pmole) | 8 μg (138 pmole) | 1 μg (14 pmole) |
| 16th | 500 | ng (15 pmole) | 8 μg (138 pmole) | 1 μg (14 pmole) |
| 17th | 500 | ng (15 pmole) | 8 μg (138 pmole) | 1 μg (14 pmole) |

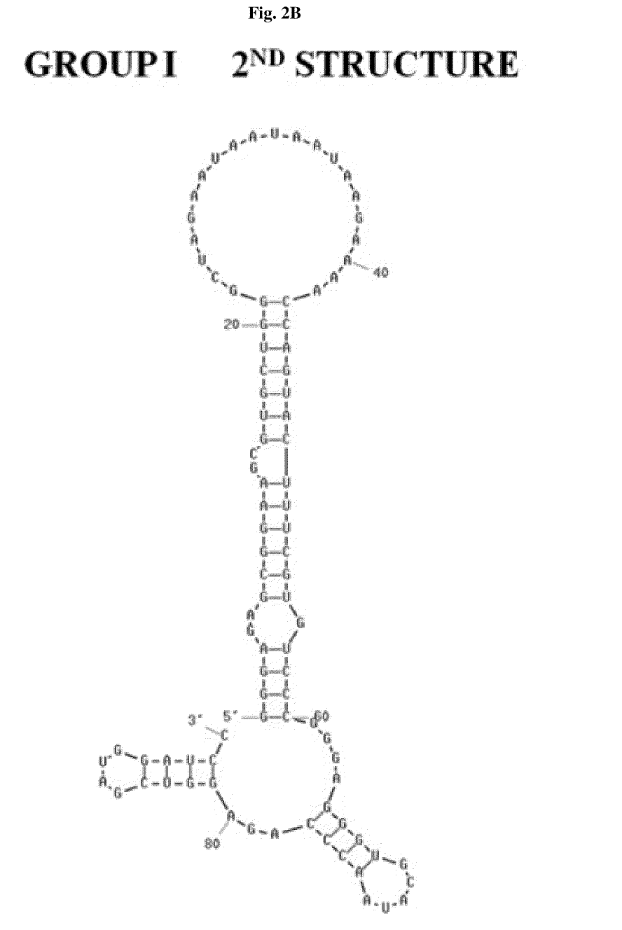

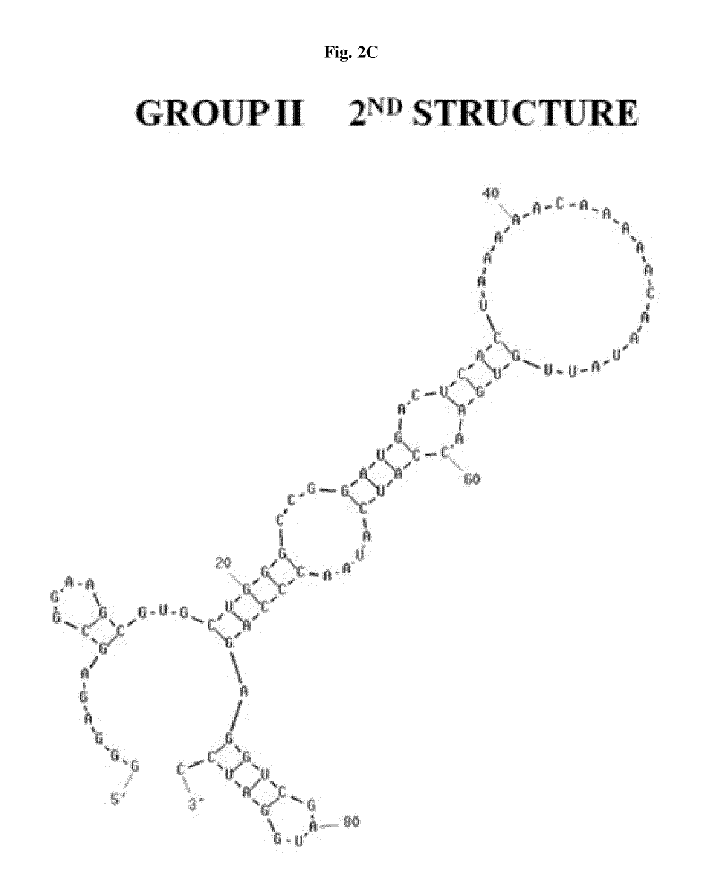

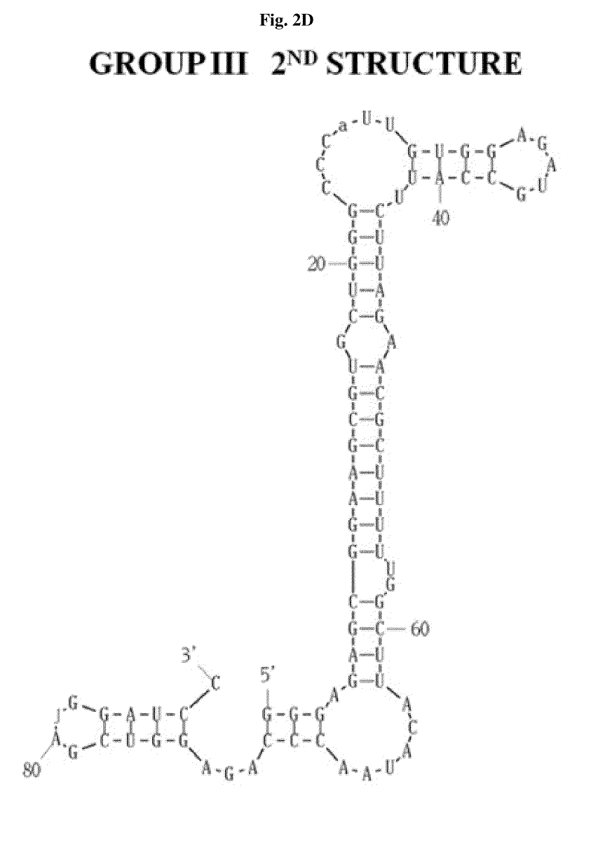

17 rounds SELEX was conducted to obtain 3 groups of RNAs with polynucleotide similarity, and the result was shown in FIG. 2A. It can be seen that the ratio of GROUP 1 RNAs are 70% or more. Secondary structures of the obtained RNAs of each GROUP were expected using mFold (Mfold web server for nucleic acid folding and hybridization prediction. Nucleic Acids Res. 31 (13), 3406-15, (2003)), and shown in FIG. 2B-2D.

### Example 5

### Measurement of Affinity of Selected RNAs to CEA

To measure the affinity of RNA to CEA, SPR analysis was performed with Biacore 2000 (GE healthcare) device. Various concentrations of RNAs (GROUP 1, 2, 3 and library in FIG. 2) were flowed on NCEA and ACEA to measure the binding affinity.

It is confirmed that GROUP 1 has the highest affinity to NCEA and shows 10 times lower KD compared to ACEA, indicating that GROUP 1 is an RNA aptamer specific to NCEA of CEA with high affinity thereto. Meanwhile, it is confirmed that GROUPs 2 and 3 have the affinity to NCEA of about 5 times (GROUP 2) or about 3 times (GROUP 3) compared to ACEA (TABLE 3).

TABLE 3
Affiniy of each GROUP to NCEA and ACEA

| | ka (1/Ms) | kd (1/s) | KA (1/M) | KD (M) | Chi2 |
|---|---|---|---|---|---|
| GROUP I to | 5.27E+05 ± | 1.68E−04 ± | 3.33E+09 ± | 3.13E−10 ± | 6.19E+00 ± |
| NCEA | 6.29E+04 | 6.58E−05 | 9.26E+08 | 8.77E−11 | 1.51E+00 |
| GROUP I to | 3.90E+05 ± | 1.25E−03 ± | 3.11E+08 ± | 3.22E−09 ± | 7.07E+00 ± |
| ACEA | 3.29E+05 | 1.04E−03 | 4.95E+06 | 5.66E−11 | 5.16E−01 |
| GROUP II | 4.09E+05 ± | 2.40E−04 ± | 1.69E+09 ± | 5.96E−10 ± | 5.21E+00 ± |
| to NCEA | 1.13E+05 | 4.10E−05 | 1.84E+08 | 6.58E−11 | 2.83E−02 |
| GROUP II | 4.22E+05 ± | 1.15E−03 ± | 3.61E+08 ± | 2.82E−09 ± | 1.07E+00 ± |
| to ACEA | 1.41E+05 | 1.70E−04 | 7.07E+07 | 5.52E−10 | 7.14E−01 |
| GROUP III | 2.71E+05 ± | 4.04E−04 ± | 6.96E+08 ± | 1.49E−09 ± | 3.82E+00 ± |
| to NCEA | 2.83E+03 | 1.10E−04 | 1.82E+08 | 3.89E−10 | 4.16E+00 |
| GROUP III | 3.39E+05 ± | 1.39E−03 ± | 2.42E+08 ± | 4.19E−09 ± | 1.94E+00 ± |
| to ACEA | 8.70E+04 | 1.20E−04 | 4.24E+07 | 7.35E−10 | 1.60E+00 |
| LIBRARY | 1.31E+05 ± | 1.62E−03 ± | 8.13E+07 ± | 1.24E−08 ± | 4.16E+00 ± |
| to NCEA | 2.83E+03 | 1.34E−04 | 5.02E+06 | 7.78E−10 | 7.99E−01 |
| LIBRARY | 9.57E+03 ± | 7.04E−04 ± | 1.33E+07 ± | 7.54E−08 ± | 4.15E+00 ± |
| to ACEA | 7.83E+03 | 5.46E−04 | 8.49E+05 | 4.95E−09 | 4.23E+00 |

The above value is measured using BIAevaluation program (which is used to analyze graph obtained from BIAcore device).

ka: concentration of analyte binding to the target per an hour

kd: concentration of analyte separating from the target per an hour

KD: equilibrium constant showing binding strength

chi2: a value showing the difference between the calculation value by the BIAevaluation program and data obtained from actual experiment, which should be 10 or less.

12 RNA aptamers of GROUP 1 shown in FIG. 2A highly specifically bind to a linkage region between N domain and A1 domain of CEA, and it can be used for an active ingredient of metastasis inhibitor of the present invention, of which polynucleotide is as shown in the following SEQ ID NO: 1 to SEQ ID NO: 12 (mutated nucleotide is shown in underline).

SEQ ID NO: 1:
GGGAGAGCGGAAGCGUGCUGGGCUAGAAUAAUAAUAAGAAAACC
AGUACUUUCGU GUCCCGGGAGGGUGCAUAACCCAGAGGUCGAUGGAUCC
SEQ ID NO: 2:
GGGAGAGCGGAAGCGUGCUGGGCUAGAAUAAUAAUAAGAAAACC
AGUACUUUCGUGUGCCGGGCGGGUCCAUAACCCAGAGGUCGAUGGAUCC
SEQ ID NO: 3
GGGAGAGCGGAAGCGUGCUGGGCUAGAAUAAUAAUAAGAAAACC
AGUACUUUCGUGUCCCGGGAGGUUCCAUAACCCAGAGGUCGAUGGAUCC
SEQ ID NO: 4:
GGGAGAGCGGAAGCGUGCUGGGCUAGAAUAAUAAUAAGAAAACC
AGUACUUUCGUGUCCCGGGAGGAGCCAUAACCCAGAGGUCGAUGGAUCC
SEQ ID NO: 5:
GGGAGAGCGGAAGCGUGCUGGGCUAGAAUAAUAAUAAGAAAACC
AGUACUUUCGUGUCCCGGGAGGACACAUAACCCAGAGGUCGAUGGAUCC
SEQ ID NO: 6:
GGGAGAGCGGAAGCGUGCUGGGCUAGAAUAAUAAUAAGAAAACC
AGAACUUUCGUGUCCCGGGAGGUUUCAUAACCCAGAGGUCGAUGGAUCC
SEQ ID NO: 7:
GGGAGAGCGGAAGCGUGCUGGGCUAGAAUAAUAAUAAGAAAACC
AGUACUUUCGCGUCCCAGGAGGGUCCAUAACCCAGAGGUCGAUGGAUCC
SEQ ID NO: 8:
GGGAGAGCGGAAGCGUGCUGGGCUAGAAUAAUAAUAAGAAAACC
AGUACUUUCGUGUCCCCGGGAGGAUUCAUAACCCAGAGGUCGAUGGAUCC
SEQ ID NO: 9:
GGGAGAGCGGAAGCGUGCUGGGCUAGAAUAAUAAUAAGAAAAAC
CAGUACUUUCGUGUCCCGGGAGAGGUACAUAACCCAGAGGUCGAUGGAU
CC
SEQ ID NO: 10:
GGGAGAGCGGAAGCGUGCUGGGCUCGAAUAAUAAUAACGAAAACC
AGUACUUUCGUGUCCCGGGAGGACCAUAACCCAGAGGUCGAUGGAUCC
SEQ ID NO: 11:
GGGAGAGCGGAAGCGUGCUGGGCUCGAAUAAUAAUAAGAAAACC
AGUACUUUCGUGUCCCGGGAGGGCCAUAACCCAGAGGUCGAUGGAUCC
SEQ ID NO: 12:
GGGAGAGCGGAAGCGUGCUGGGCUAGAAUAAUAAUAAGAAAACC
AGUACUUUCGUGUCCCGGGAGGGCCAUAACCCAGAGGUCGAUGGAUCC

### Example 6

### Optimization of RNA Aptamer Specifically Binding to CEA

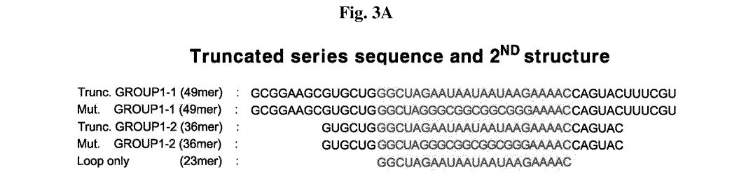

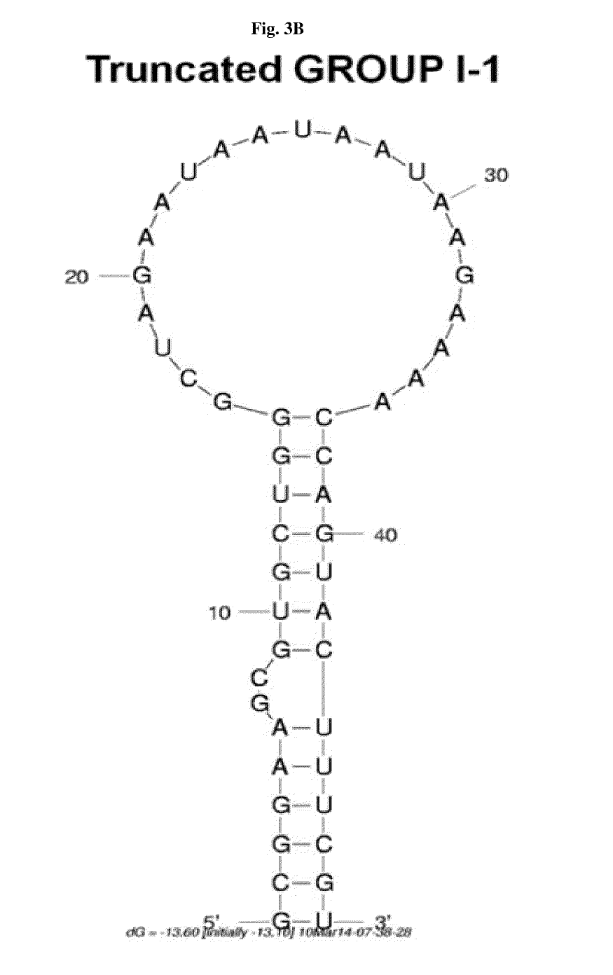

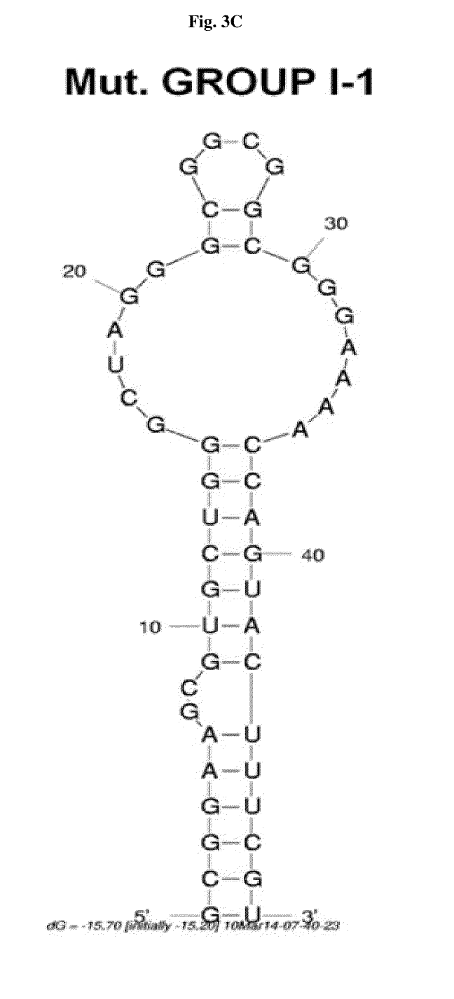

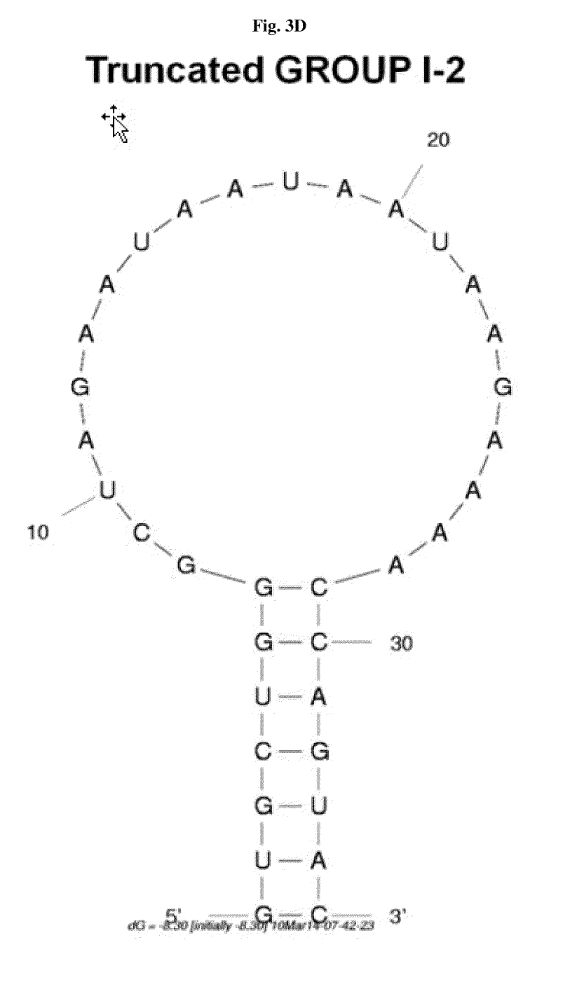

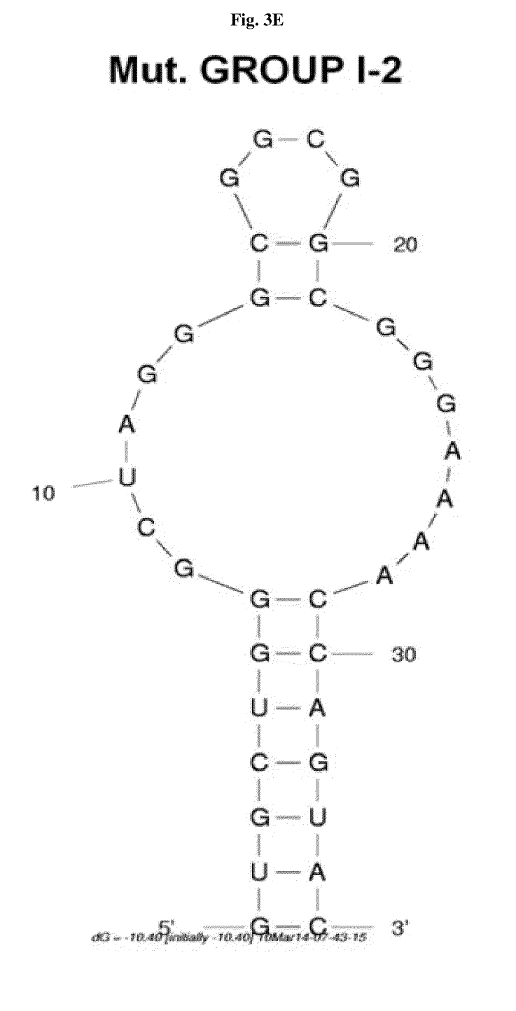

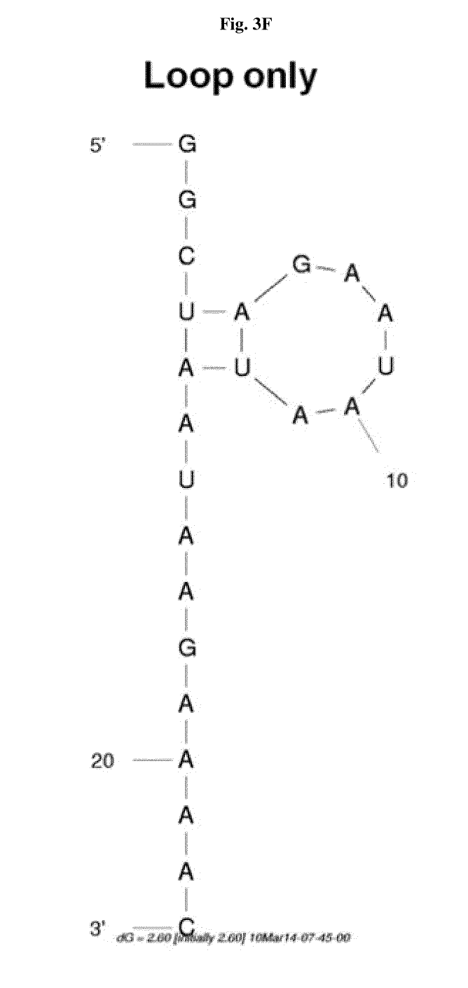

The length of GROUP1 showing the highest affinity in the above experiment was optimized so as to be suitable for chemical synthesis. And, mutant series wherein a Loop part that is expected to be responsible for binding to a target protein, NCEA, is mutated, were constructed as follows (See FIGS. 3A-3F).

Truncated GROUP 1-1 (49mer):
(SEQ ID NO: 13)
GCGGAAGCGUGCUGGGCUAGAAUAAUAAUAAGAAAACCAGUACUUUCGU
Truncated GROUP 1-2 (35mer):
(SEQ ID NO: 14)
GUGCUGGGCUAGAAUAAUAAUAAGAAAACCAGUAC
Mutant GROUP 1-1 (49mer):
(SEQ ID NO: 15)
GCGGAAGCGUGCUGGGCUAGGGCGGCGGCGGGAAAACCAGUACUUUCGU
Mutant GROUP 1-2 (35mer):
(SEQ ID NO: 16)
GUGCUGGGCUAGGGCGGCGGCGGGAAAACCAGUAC
Loop only (23mer):
(SEQ ID NO: 17)
GGCUAGAAUAAUAAUAAGAAAAC
(Nucleotide in italic: loop part corresponding
to SEQ ID NO: 17
Nucleotide in underline: mutated part)

### Example 7

### Measurement of Affinity of RNA Aptamer to CEA

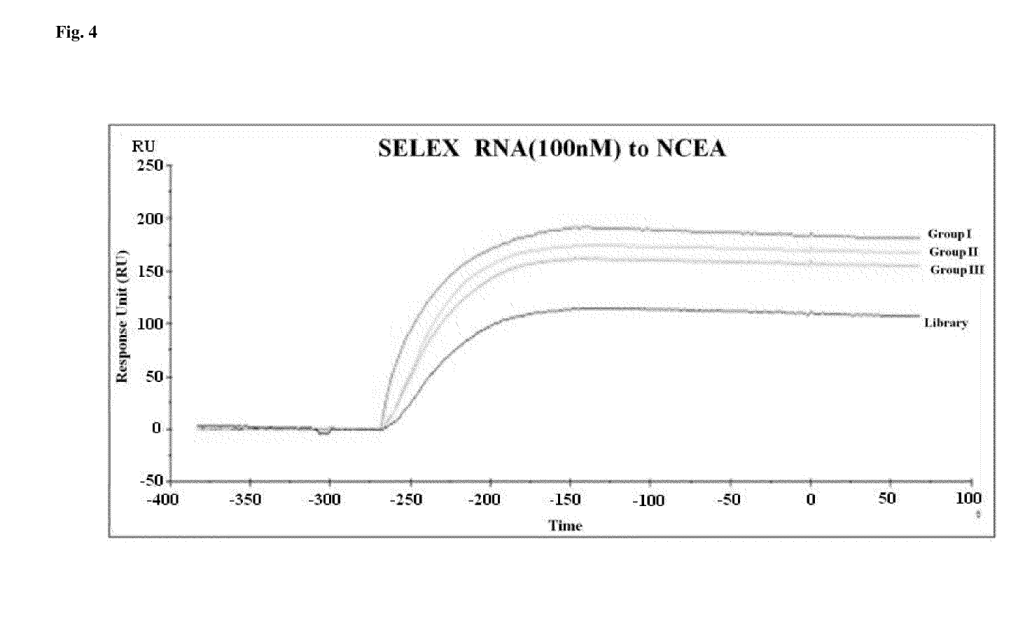

To measure the affinity of RNA aptamer to CEA, SPR analysis was performed with Biacore 2000 (GE healthcare) device. Various concentrations of RNAs (Trunc. GROUP 1-1 & 1-2 and Mutant GROUP 1-1 & 1-2, Loop-only, Library) were flowed on NCEA and ACEA to measure the binding affinity, which is shown in the following TABLE 4 and FIG. 4.

TABLE 4
Affinity of RNA aptamer to NCEA and ACEA

| | ka (1/Ms) | kd (1/s) | KA (1/M) | KD (M) | Chi2 |
|---|---|---|---|---|---|
| 19-1 NCEA | 7.0E+05 ± | 5.4E−04 ± | 1.3E+09 ± | 7.7E−10 ± | 5.5E+00 ± |
| | 1.2E+05 | 6.5E−05 | 5.7E+07 | 3.3E−11 | 3.1E+00 |
| m19-1 | 7.6E+00 ± | 1.0E−05 ± | 7.6E+05 ± | 1.3E−06 ± | 2.3E+00 ± |
| NCEA | 1.6E−01 | 0.0E+00 | 1.6E+04 | 2.8E−08 | 3.1E−01 |
| 19-2 NCEA | 3.2E+05 ± | 1.3E−03 ± | 2.5E+08 ± | 4.0E−09 ± | 2.3E+00 ± |
| | 2.4E+05 | 9.8E−04 | 5.7E+06 | 9.2E−11 | 1.9E+00 |
| m19-2 | 4.5E+01 ± | 1.8E−05 ± | 2.4E+06 ± | 4.2E−07 ± | 5.1E−01 ± |
| NCEA | 3.4E+01 | 1.1E−05 | 3.7E+05 | 6.6E−08 | 5.6E−02 |
| library | 4.8E+00 ± | 1.0E−05 ± | 4.8E+05 ± | 2.2E−06 ± | 1.2E+00 ± |
| NCEA | 1.4E+00 | 0.0E+00 | 1.4E+05 | 6.3E−07 | 8.9E−01 |
| loop-only | 4.2E+00 ± | 1.0E−05 ± | 4.1E+05 ± | 2.5E−06 ± | 3.2E+00 ± |
| NCEA | 1.3E+00 | 0.0E+00 | 1.3E+05 | 7.8E−07 | 2.8E+00 |
| 19-1 ACEA | 7.6E+03 ± | 4.6E−03 ± | 1.7E+06 ± | 6.0E−07 ± | 7.6E+00 ± |
| | 2.1E+03 | 1.7E−03 | 1.7E+05 | 6.0E−08 | 4.0E−01 |
| m19-1 | 3.6E+02 ± | 2.7E−04 ± | 1.3E+06 ± | 7.9E−07 ± | 4.6E−01 ± |
| ACEA | 5.0E+02 | 3.6E−04 | 1.3E+05 | 8.0E−08 | 2.3E−01 |
| 19-2 ACEA | 1.9E+03 ± | 1.1E−03 ± | 1.7E+06 ± | 5.9E−07 ± | 8.3E+00 ± |
| | 9.7E+02 | 5.0E−04 | 9.2E+04 | 3.3E−08 | 9.8E−01 |
| m19-2 | 1.6E+01 ± | 1.0E−05 ± | 1.6E+06 ± | 6.3E−07 ± | 1.6E−01 ± |
| ACEA | 2.1E+00 | 0.0E+00 | 2.1E+05 | 8.1E−08 | 1.2E−01 |
| library | 2.3E+00 ± | 1.0E−05 ± | 2.3E+05 ± | 4.5E−06 ± | 7.1E−01 ± |
| ACEA | 3.7E−01 | 0.0E+00 | 3.6E+04 | 7.1E−07 | 2.5E−01 |
| loop-only | 9.3E+00 ± | 1.0E−05 ± | 9.2E+05 ± | 1.1E−06 ± | 5.1E−01 ± |
| ACEA | 4.0E−01 | 0.0E+00 | 4.0E+04 | 4.9E−08 | 3.4E−01 |

As shown in TABLE 4, it is confirmed that GROUP 1-1 has the highest affinity to NCEA, and shows 780 times lower KD compared to ACEA, indicating that the optimized GROUP 1-1 is specific to NCEA of CEA with strong affinity and can be used as RNA aptamer of the present invention. GROUP 1-2 of smaller size, although has rather decreased affinity to NCEA, has superior affinity to library RNA pool or loop-only. Meanwhile, it is confirmed that library RNA pool or loop-only does not bind to NCEA. It is also confirmed that mutant GROUPs 1-1 & 1-2 with mutated loop parts show weakened binding to NCEA or do not bind thereto. The results mean that a loop part of RNA aptamer is responsible for binding to NCEA.

In conclusion, RNA aptamers of the optimized GROUP 1-1 (SEQ ID NO: 13) and GROUP 1-2 (SEQ ID NO: 14) specifically bind to a linkage region between N domain and A1 domain of CEA, and thus can be used as an active ingredient of the metastasis inhibitor of the present invention.

### Example 8

### Mass Production of RNA Aptamer Specific to Metastatic Domain of CEA

To prove that the CEA-specific RNA aptamer is a CEA-mediated metastasis inhibitor, the optimized Truncated GROUP 1-1 (SEQ ID NO: 13) and its Mutant (SEQ ID NO: 15) were mass-produced through chemical synthesis.

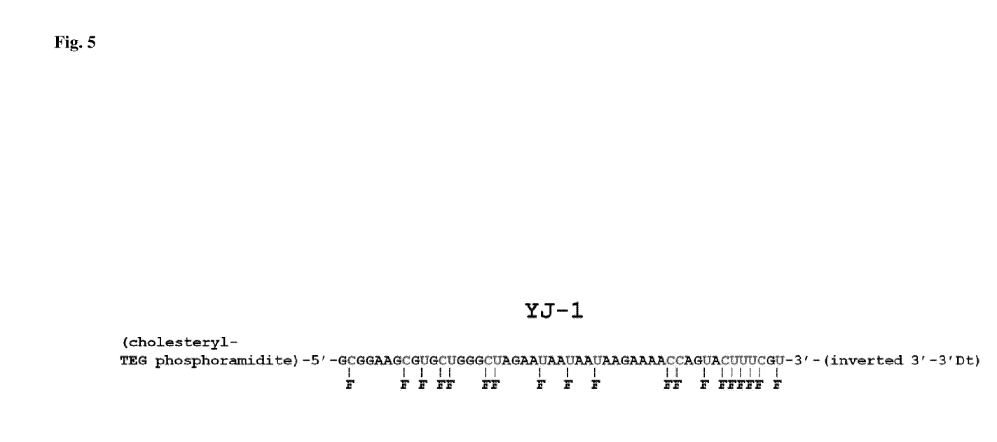

At this time, to increase in vivo aptamer availability and prevent RNase attack, cholesterol was attached to 5′ end of the RNA aptamer and inverted dT (idT) was attached to 3′ end. And, 2′ of each pyrimidine nucleotide was substituted with fluoro group (see FIG. 5). The synthesized and modified RNA aptamers are designated as YJ-1 (corresponding to SEQ ID NO: 13) and Mutant (corresponding to SEQ ID NO: 15), respectively.

To synthesize the modified aptamer, RNA aptamer (YJ-1) and its mutant RNA aptamer were synthesized with 1 mmol scale using idT CPG (solid support, SAMCHULLY PHARM. CO., LTD.). At this time, cholesterol group was attached to 5′ end using Cholesteryl TEG amidite (1-dimethoxytrityloxy-3-O-(N-cholesteryl-3-aminopropyl)-triethyleneglycol-glyceryl-2-O-(2-cyanoethyl)-(N,Ndiisopropyl)-phosphoramidite). The synthesis of cholesterol-attached aptamer conjugate was examined with polyacrylamide gel electrophoresis, HPLC (Agilent 1100, Agilent technologies) and MALDI-TOF (Autoflex MALDI-TOF Mass Spectrometer, Bruker Daltonics), and it was precipitated and desalted with CentriSep (ABI applied biosystems), and finally dissolved in water.

### Example 9

### Inhibition of CEA-Dependent Cancer Cell Aggregation by RNA Aptamer

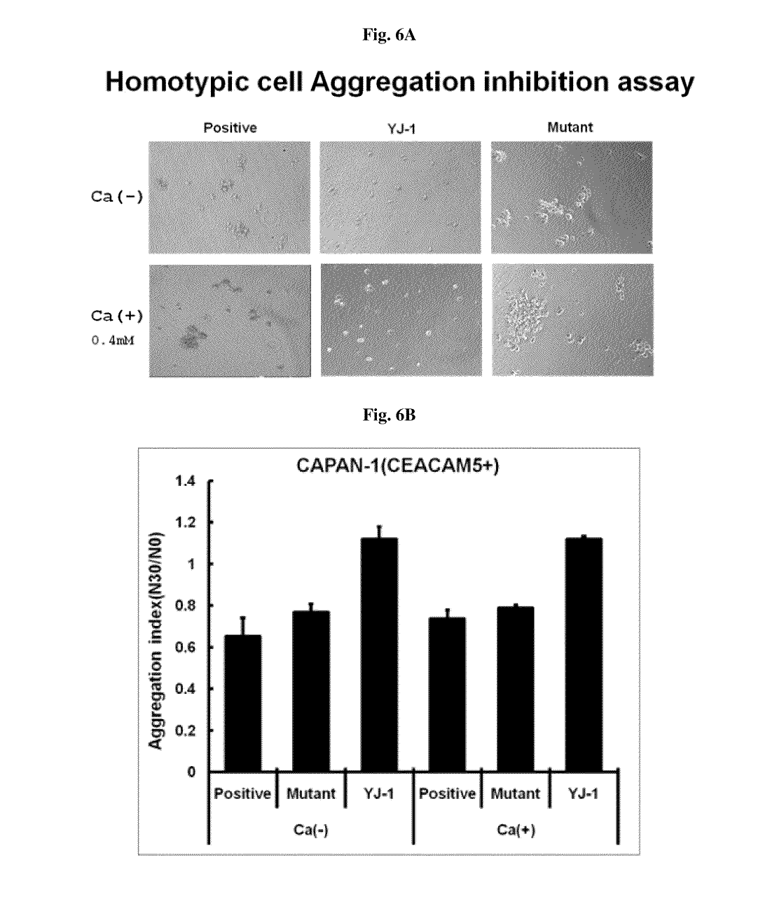

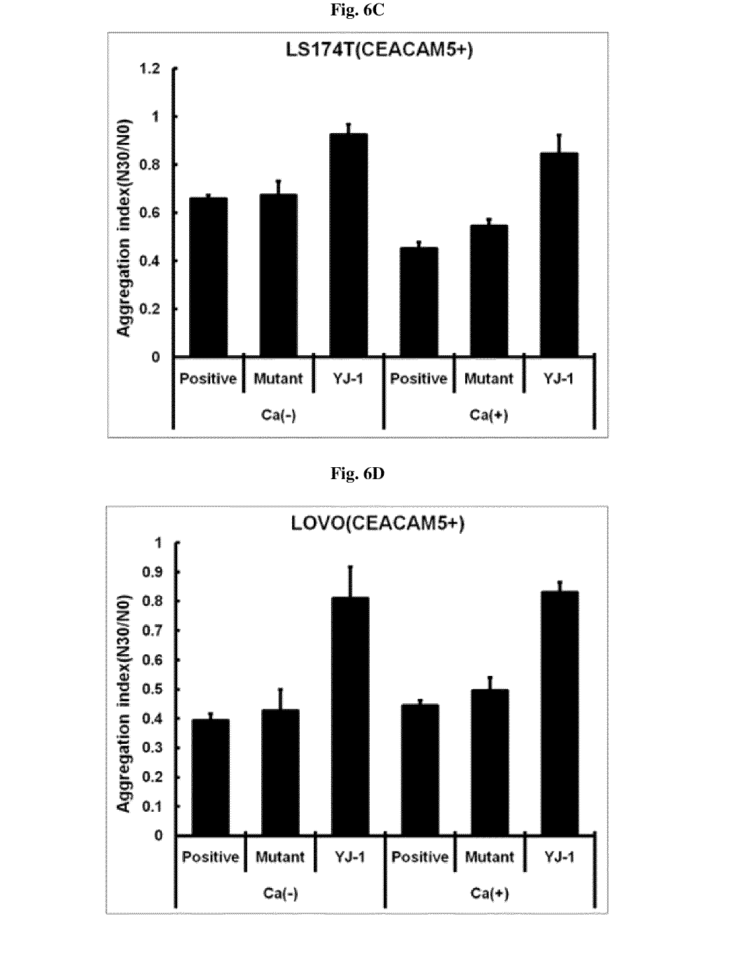

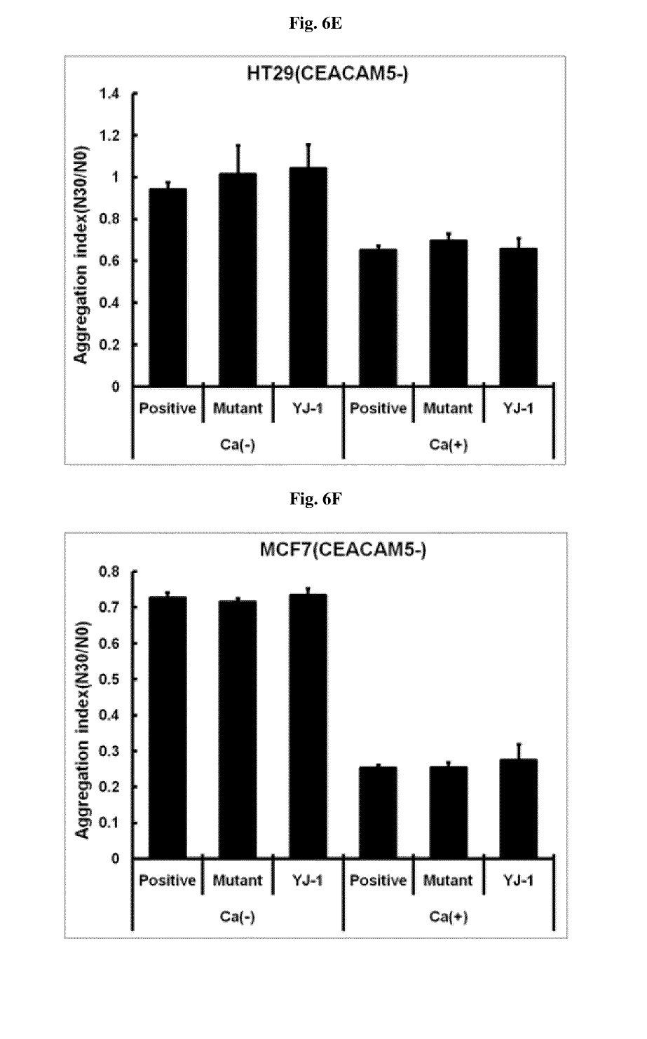

CEA-mediated cell aggregation assay was performed to measure aggregation index to see if RNA aptamer (YJ-1) inhibits CEA-mediated cell aggregation between CEA-positive cells (See FIG. 6).

CEA-positive cell lines, i.e., LS174T (CL-188 ATCC(American Type Culture Collection)), LoVo(CCL-229 ATCC), and CAPAN-1 (HTB-79 ATCC) cell lines, and CEA-negative cell lines, i.e., HT29 (HTB-38 ATCC) and MCF7 (HTB-22 ATCC) cell lines, were incubated with each suitable medium (LS174T, MCF7: MEM(Minimum Essential Medium)/10% FBS/1% Antibiotic/Antimycotic Solution; CAPAN-1: RPMI 1640/MEM/20% FBS/1% Antibiotic/Antimycotic Solution; LoVo, HT29: DMEM(Dulbecco's Modified Eagle's Media)/10% FBS/1% Antibiotic/Antimycotic Solution, Thermo Fisher Scientific Inc. (HyClone)) at 37° C., 5% CO₂ (2.5×10⁶ cells/24 hrs).

To confirm the inhibition of CEA-dependent cancer cell aggregation, the above-obtained cells were subcultured once, and separated from the culture dish with Non-enzymatic cell dissociation buffer (sigma), and then, the number of cells was determined with Hemocytometer (1×10⁶ cells/ml). The obtained cells were treated with each 10 m/ml of the above-selected RNA aptamers of SEQ ID NO: 13 and SEQ ID NO: 15, and each cell was suspended in 0.4 mM Ca²⁺ treated PBS and non-treated PBS, and then, introduced in a 24 well plate and shaken at 37° C. for 30 minutes at 80 rpm, and then, fixed with 5% glutaraldehyde, and the degree of aggregation was indicated by aggregation index (N30/N0; NO: the number of single cell to total number of aggregated cells at 0 minute, N30: the number of single cell to total number of aggregated cells after 30 minutes of reaction).

The results were shown in FIGS. 6A-F. FIG. 6A is a photo representatively showing whether or not cell aggregation degree is inhibited depending on the presence of RNA aptamer (YJ-1 or Mutant), and 6B-6F are graphs showing aggregation index (N30/N0) derived by counting 10 photos as the above for each treatment group.

As shown in FIG. 6, in CEA-positive cells, i.e., LS174T, Lovo, and CAPAN cells, cell aggregation is effectively inhibited by RNA aptamer (YJ-1, SEQ ID NO: 13) in a calcium-independent manner, while in CEA-negative cells, i.e., MCF7 and HT-29 cells, cell aggregation increased in a calcium-dependent manner, irrespective of the presence of RNA aptamer (YJ-1, SEQ ID NO: 13).

### Example 10

### Inhibition of CEA-Dependent In Vitro ECM Adhesion by RNA Aptamer

To examine whether the selected RNA aptamer can inhibit the adhesion of CEA to extracellular matrix (ECM) component protein, the degree of adhesion to 5 ECM (extracellular matrix) proteins (fibronectin, vitronectin, laminin, collagen I, and collagen IV) were measured. At adhesion reaction of CEACAM5 expressing cancer cell and non-expressing cancer cell with ECM protein, RNA aptamer (300 nM) was incubated together, and then, cancer cells adhered to ECM protein were dyed with a staining solution (0.2% crystal violet in 10% ethanol) and measured to examine the adhesion degree.

More specifically, CEA-positive or negative cells were bound to a plate (Chemicon International Inc. (CytoMatrix™ (5) SCREEN KIT)) coated with the 5 ECM protein (fibronectin, vitronectin, laminin, collagen I and collagen IV). For binding reaction, CEA-positive cell lines, i.e., LS174T, LoVo, SW480 (CCL-228 ATCC) and CAPAN-1 cells lines, and CEA-negative cell lines, i.e., HT29, MCF7, and NIH-3T3 (CRL-1658 ATCC) cell lines were incubated with each appropriate medium (LS174T, MCF7:MEM/10% FBS/1%, Antibiotic/Antimycotic Solution, CAPAN-1: RPMI 1640/MEM/20% FBS/1% Antibiotic/Antimycotic Solution, SW480, LoVo, HT29, NIH-3T3: DMEM/10% FBS/1% Antibiotic Antimycotic Solution) at 37° C. 5% CO₂ (2.5×10⁶ cells/24 hrs), separated from the culture dish using Non-enzymetic cell dissociation buffer, and then, the number (1×10⁶ cells/ml) of cells was determined with Hemocytometer.

The obtained cells were not treated with RNA aptamer, or treated with RNA aptamer (YJ-1: 10 μg/ml) of SEQ ID NO: 13 or Mutant Aptamer (10 m/ml) of SEQ ID NO: 15, and introduced into a well coated with each ECM protein, and then, incubated at 37° C. 5% CO₂ for 30 minutes, and non-bound cells were washed with PBS containing Ca²⁺ and Mg²⁺3 times. And then, remaining cells were stained with 0.2% crystal violet (included in CytoMatrix™ (5) SCREEN KIT), and eluted by Solubilization Buffer (included in CytoMatrix™ (5) SCREEN KIT) and measured at 570 nm (microplate reader 550 Biorad).

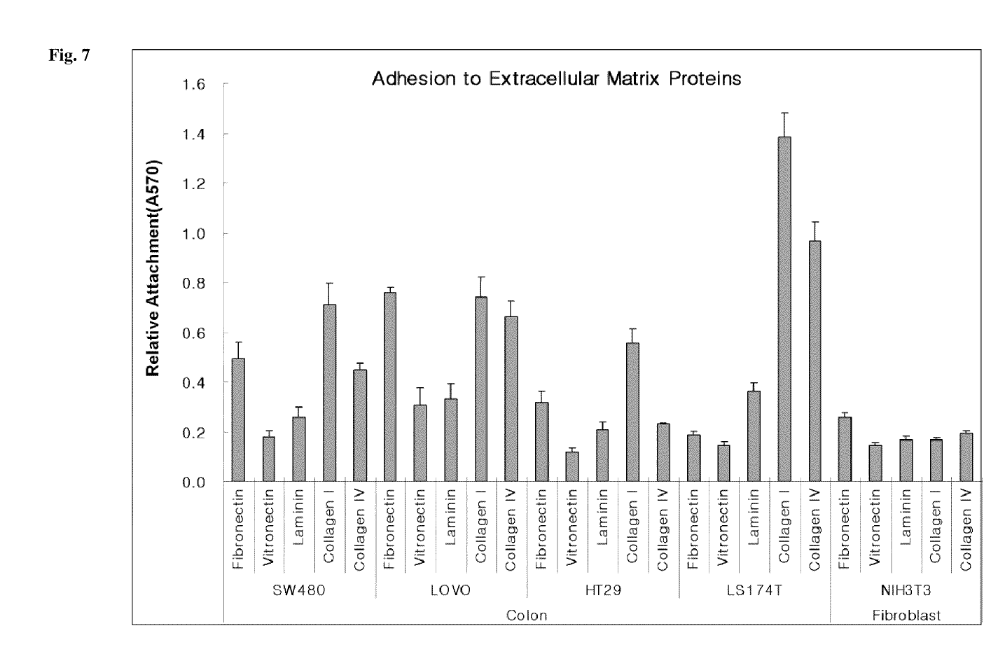

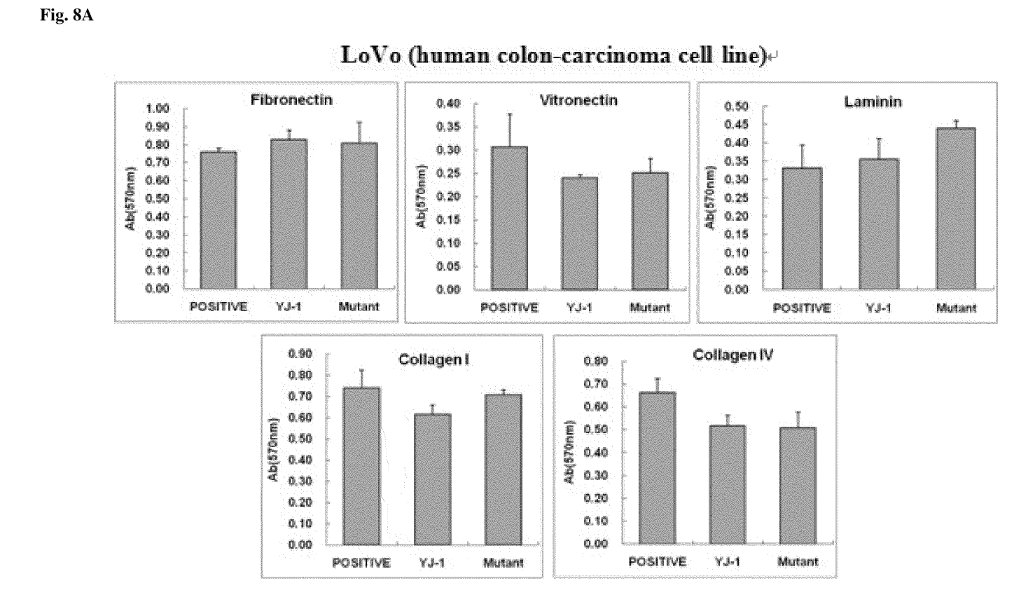

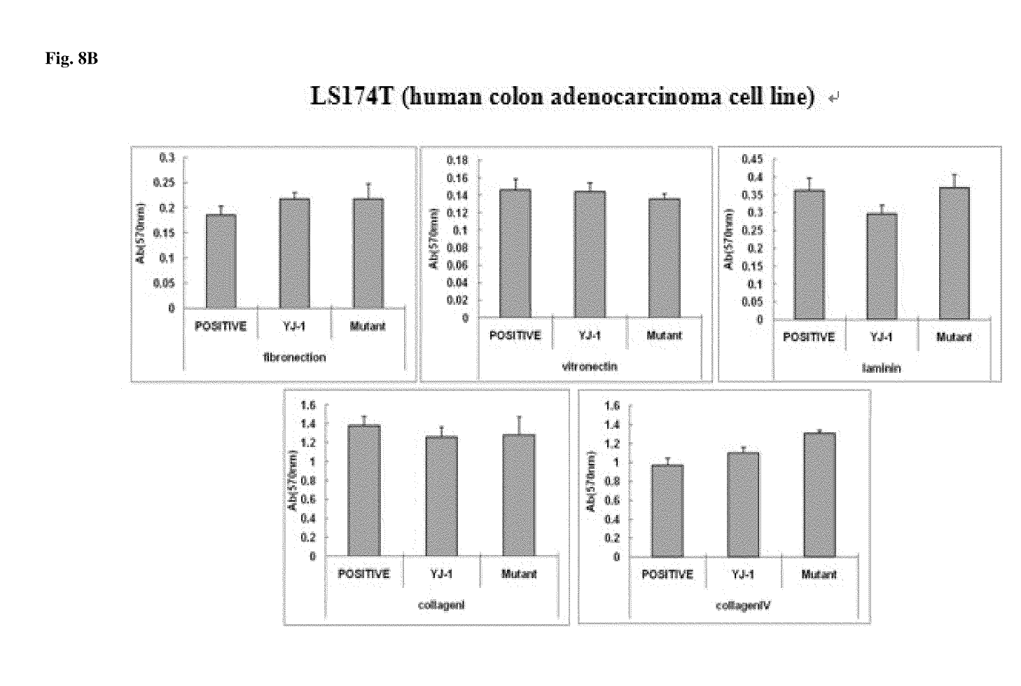

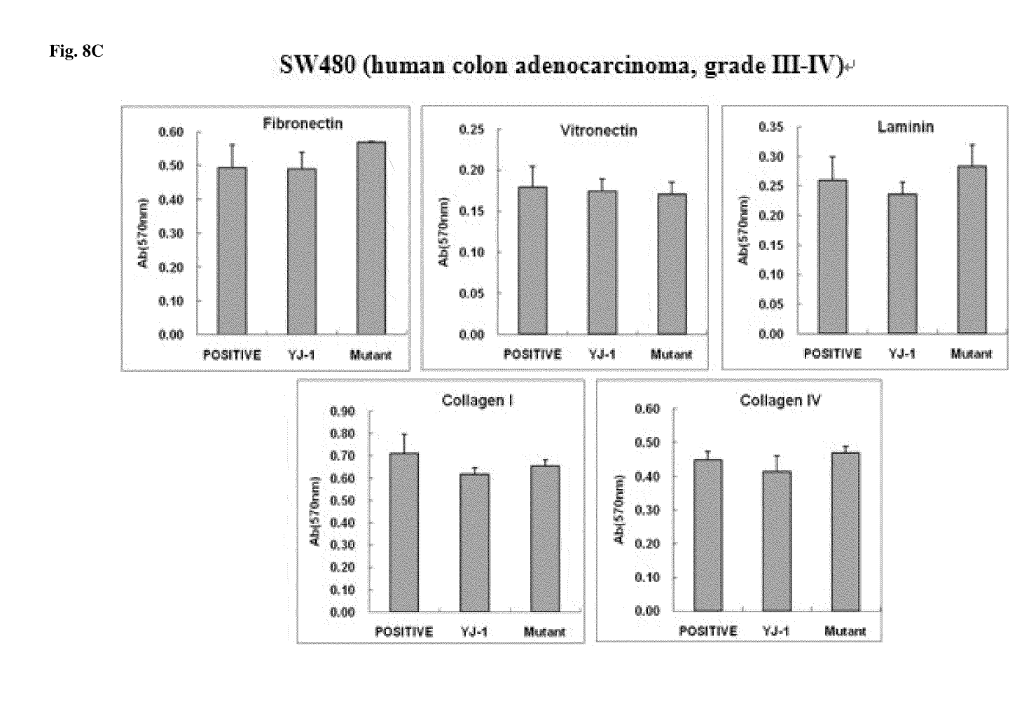

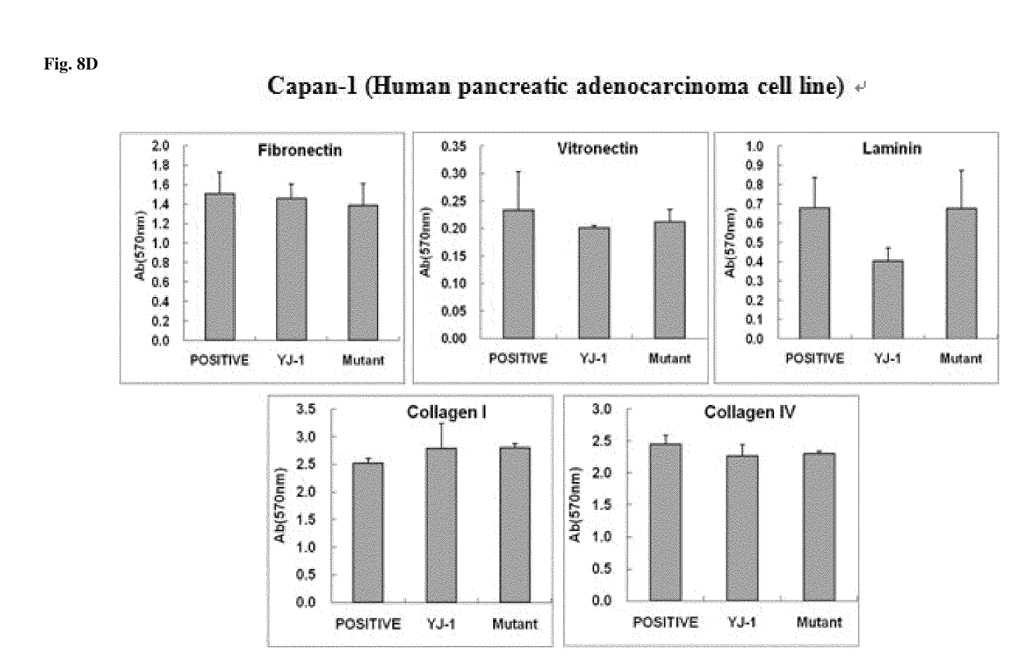

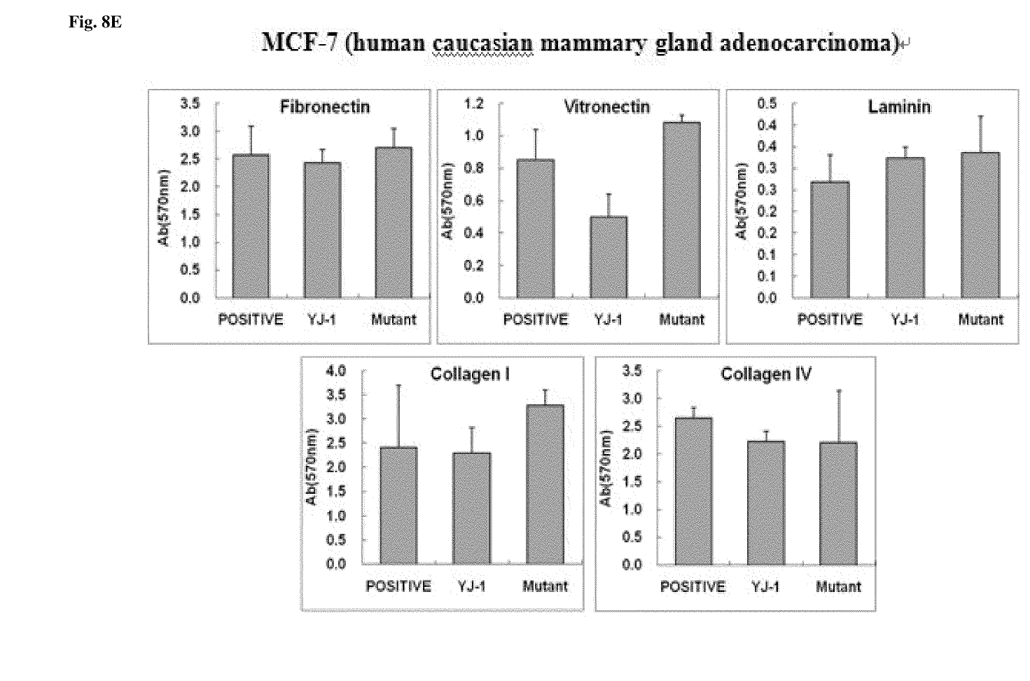

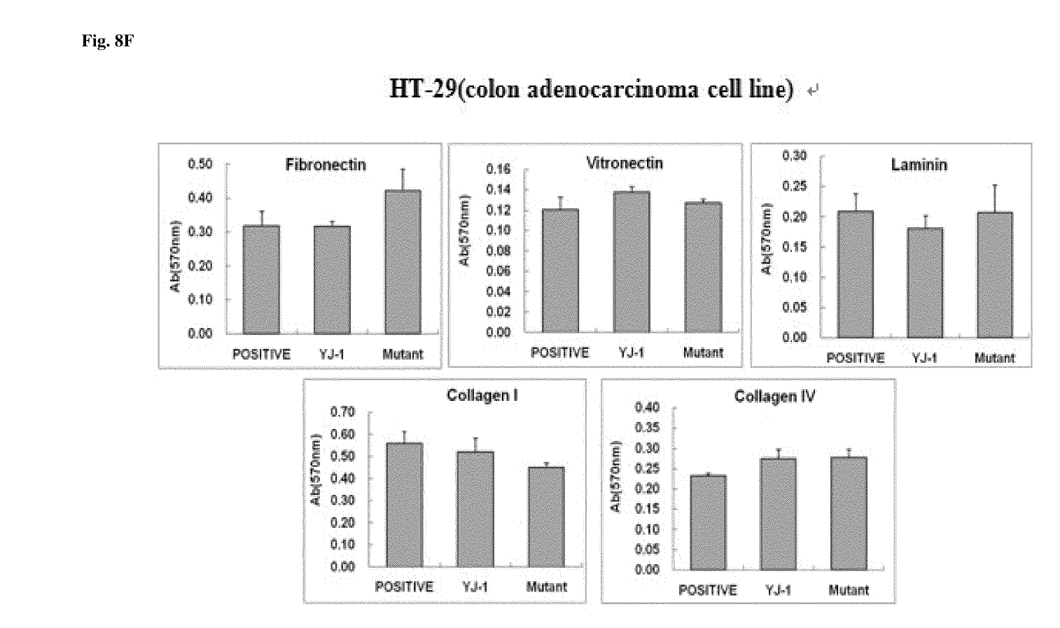

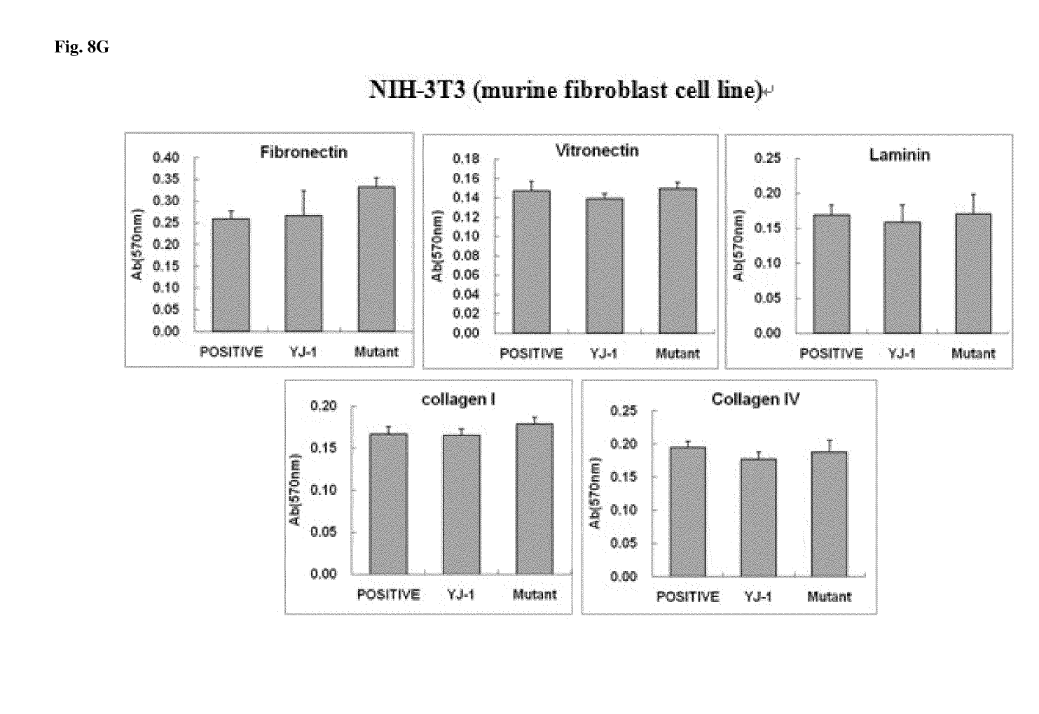

Relative adhesion degree of RNA aptamer non-treated cells is shown in FIG. 7, and the adhesion degrees of RNA aptamer treated cells are shown in FIG. 8A-8G (A-D: CEA-positive cell lines, E-G: CEA-negative cell lines). As shown in FIGS. 7 and 8, it is confirmed that the adhesion of most cancer cells to ECM proteins is not inhibited by RNA aptamer (YJ-1, SEQ ID NO: 13), while the adhesion of some CEA-positive cancer cells, CAPAN-1 and LS174T, to one of ECM proteins, Laminin is inhibited.

### Example 11

### Inhibition of CEA Dependent In Vitro Invasion by RNA Aptamer

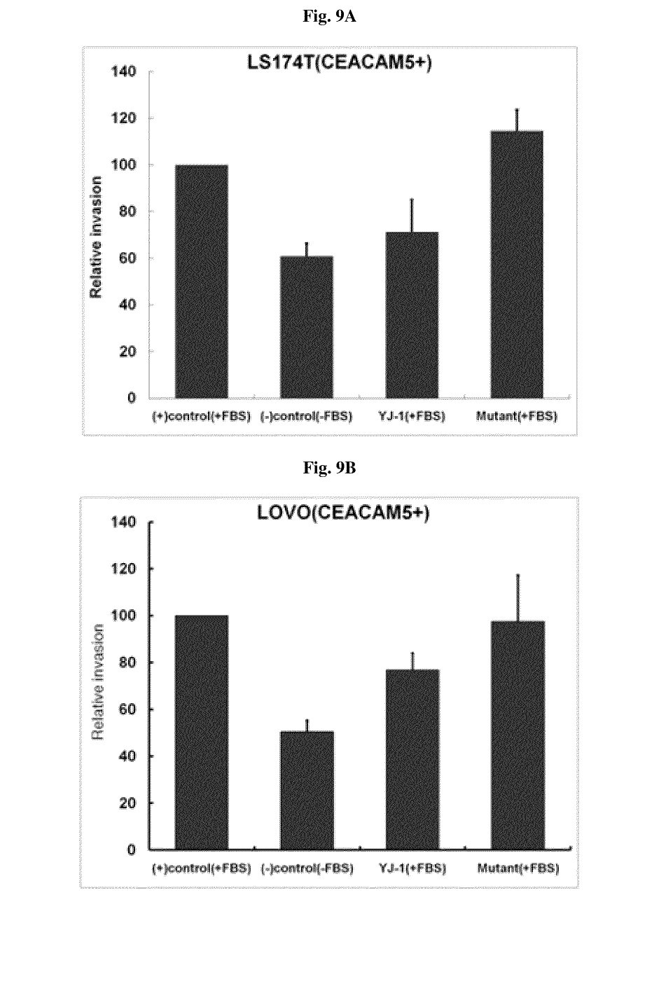

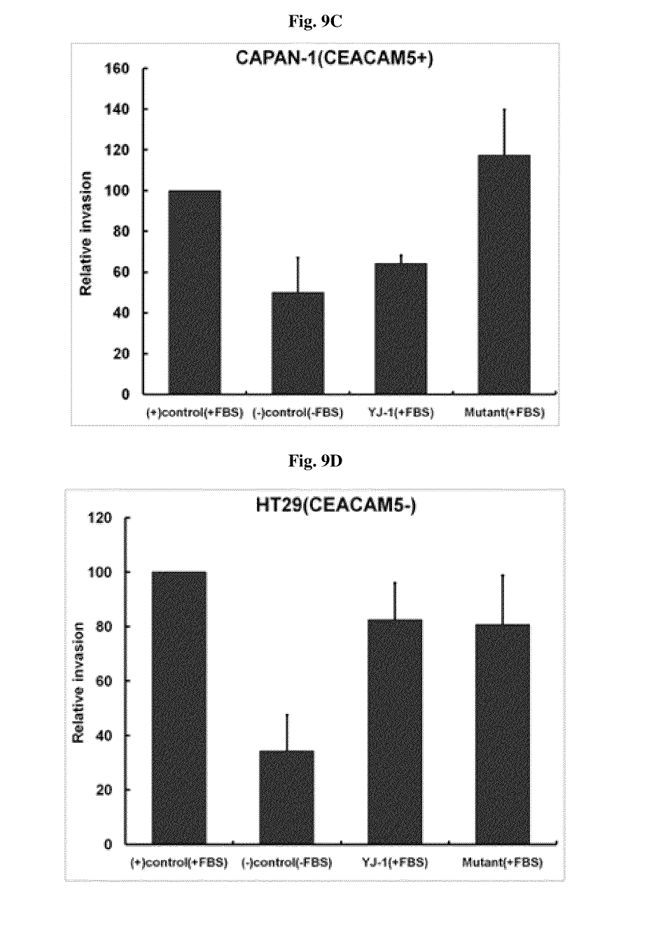

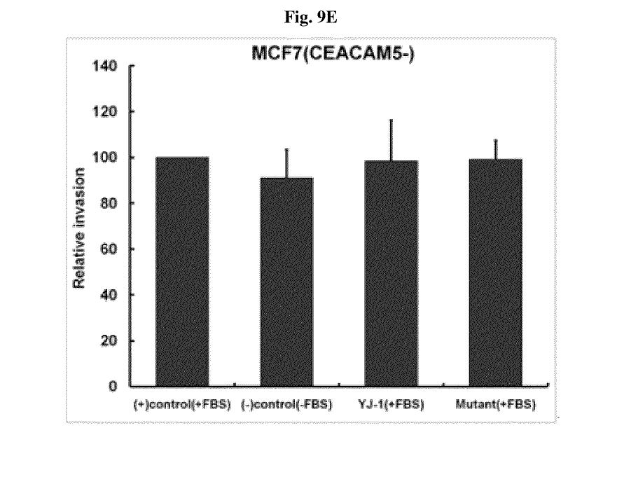

To confirm the inhibition function of RNA aptamer in the process of invasion in the steps of metastasis, Collagen-Based Cell Invasion inhibition Assay was performed (See FIG. 9).

To confirm inhibition of CEA dependent in vitro invasion by selected RNA aptamer, the cells incubated under the incubation conditions described in Example 10 were starved for 24 hours, and separated from culture dish using Non-enzymetic cell dissociation buffer, and then, washed with quenching medium (serum-free DMEM containing 5% BSA, Serum-free DMEM (HyClone), BSA (sigma)), and the number of cells was determined with Hemocytometer (1×10⁶ cells/ml).

And then, the cells were treated with RNA aptamer (SEQ ID NO: 13, 10 m/ml) or Mutant aptamer (SEQ ID NO: 15, 10 m/ml), introduced into collagen (BD biosciences) coated inserts, and incubated in a 37° C. 5% CO₂ incubator for 48 hours, and then, invaded cells were dyed and measured at 490 nm (microplate reader 550 Biorad).

The results are shown in FIGS. 9A-9E. As shown in FIGS. 9A-9E, it is confirmed that in CEA-positive cells, i.e., LS174T, CAPAN-1 and LoVo cells, RNA aptamer (YJ-1, SEQ ID NO: 13) specifically inhibits invasion of cancer cell, while in CEA-negative cells, i.e., HT29 and MCF7 cells, RNA aptamer (YJ-1, SEQ ID NO: 13) does not have influence on invasion of cancer cell.

### Example 12

### Inhibition of CEA Dependent Liver Metastasis of Colon Cancer by RNA Aptamer in Metastasis Animal Model

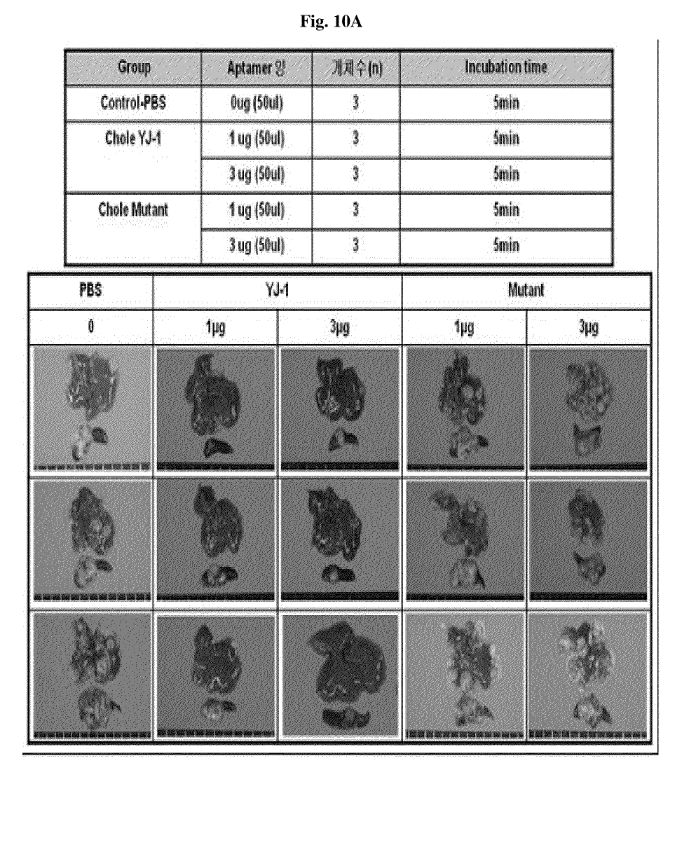

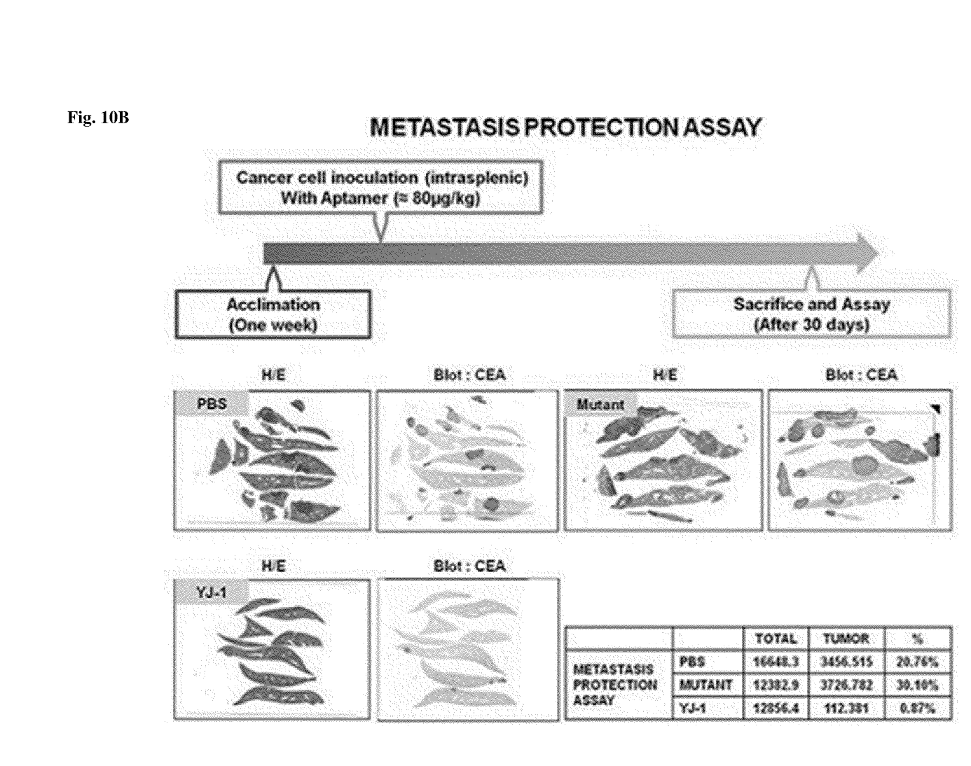

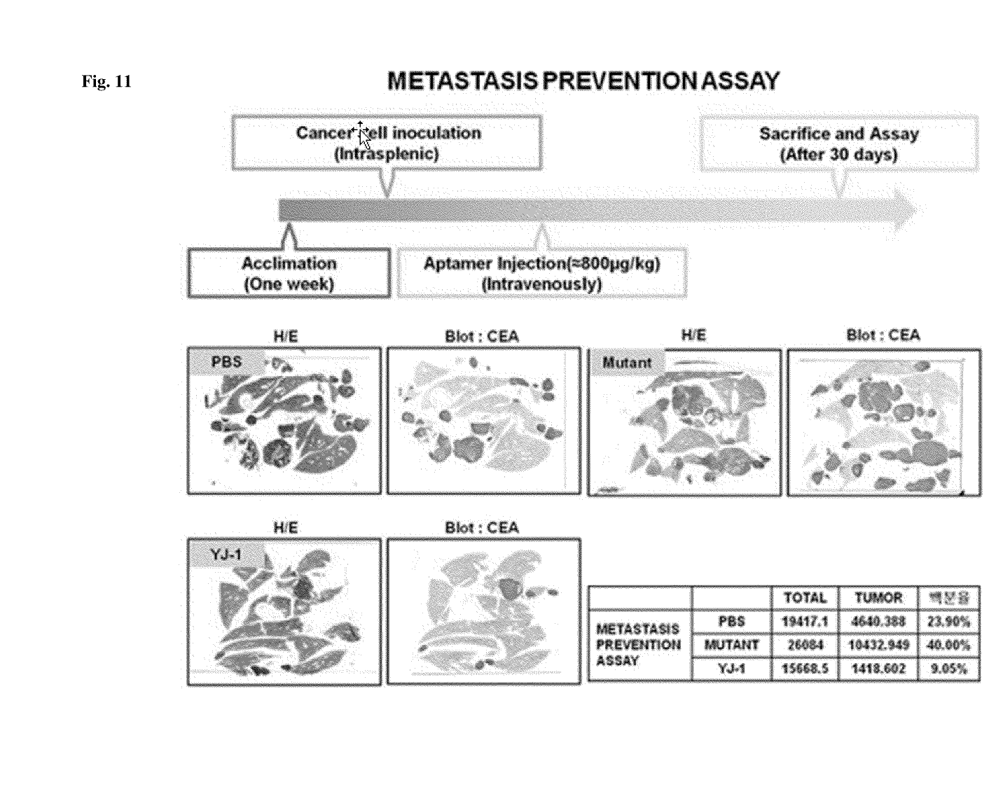

About 6 week-old male nude mice (Balb/CAnN/CriBg-nu/nu, ORIENT.CO.LTD) received intrasplenic injection of colon cancer cell line, i.e., LS174T cells (1×10⁶), and used as an animal model of liver metastasis. CEA-positive cell, i.e., LS174T colon cancer cell (CL-188 ATCC) and RNA aptamer (YJ-1, SEQ ID NO: 13) were incubated, and then, the reacted cells were intrasplenically injected into the mice to examine whether the formation of metastatic tumor to the liver is inhibited by RNA aptamer (YJ-1) in the metastasis mouse model (See FIG. 10, and FIG. 11).

For a metastasis inhibition assay, about 6 week-old male nude mouse was acclimated for 1 week, and then, CEA-positive cell, i.e., LS174T cell line (2×10⁶ cells), and selected RNA aptamer (YJ-1, ≈80 μg/kg) or its mutant aptamer (mutant YJ-1, ≈80 μg/kg) were incubated at 37° C. for 5 minutes, and intrasplenically injected. After 30 days, the mouse was sacrificed, and the degree of metastasis was analyzed and shown in FIGS. 10A and 10B.

For a metastasis prevention assay, about 6 week-old male nude mouse was acclimated for 1 week, and then, CEA-positive cell, i.e., LS174T cell line (2×10⁶ cells) were intrasplenically injected, and after 10 days, selected RNA aptamer (YJ-1, ≈80 μg/kg) or its mutant aptamer (mutant YJ-1, ≈80 μg/kg) was intravenously injected through the tail vein of the mouse, and after 30 days, the mouse was sacrificed, and the degree of metastasis was analyzed by Immunohistochemistry (Ramos-Vara, JA (2005). “Technical Aspects of Immunohistochemistry”. Vet Pathol 42 (4): 405-426) and shown in FIG. 11

As shown in FIG. 10, it is confirmed that when CEA aptamer (SEQ ID NO: 13) is incubated, liver metastasis remarkably decreases compared to mutant aptamer (SEQ ID NO: 15). And as shown in FIG. 11, it is confirmed by Immunohistochemistry that in a metastasis prevention assay wherein CEA-positive cell, i.e., LS174T colon cancer cells are intravenously injected first, and, after 10 days, RNA aptamer (YJ-1) or Mutant (mutant YJ-1) is intravenously injected, RNA aptamer (YJ-1) also specifically inhibits liver metastasis of colon cancer cell, compared to Mutant (mutant YJ-1).

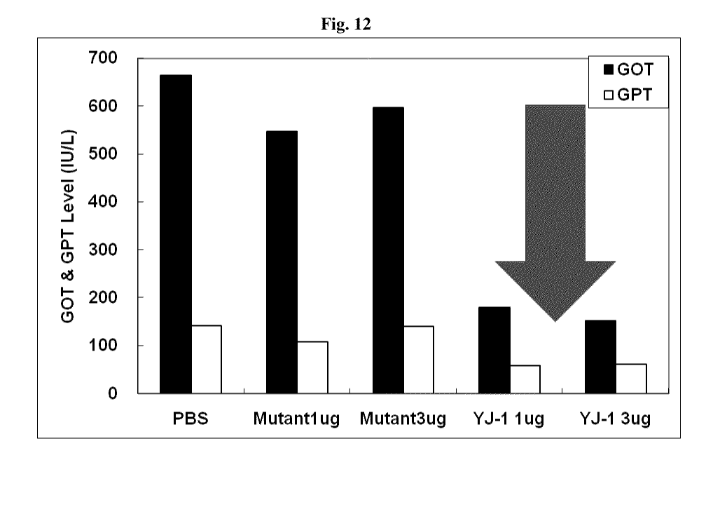

It is also confirmed with an automation device, Toshiba TBA-200FR, that in vivo RNA aptamer (YJ-1) treatment does not cause toxicity in liver tissue of mice (FIG. 12).

12. Inhibition of CEA Dependent In Vitro Migration by RNA Aptamer

To confirm the inhibition function of RNA aptamer (YJ-1) in the process of migration in the steps of metastasis, Cancer cell migration inhibition Assay was performed. Migration assay kit(Oris™ Cell Migration Assay kit, Platypus Technologies) was used, and the method was as follows. After determining the number of incubated cells with Hemocytometer (5×10⁴ cells/ml), they were seeded into a well. After incubating confluently, the cells were starved for 16 hours, and the kit insert was removed, and then, the cells were treated with selected RNA aptamer (for example, YJ-1 aptamer: 10 μg/ml) or its Mutant aptamer (for example, Mutant YJ-1 aptamer: 10 μg/ml) and incubated in a 37° C. 5% CO2 incubator for 24 hours, and then, cells which moved to the center were dyed with Calcein AM Fluorescent Dye (4 ug/ml, BD Biosciences) and measured at 490 nm (microplate reader 550, Biorad).

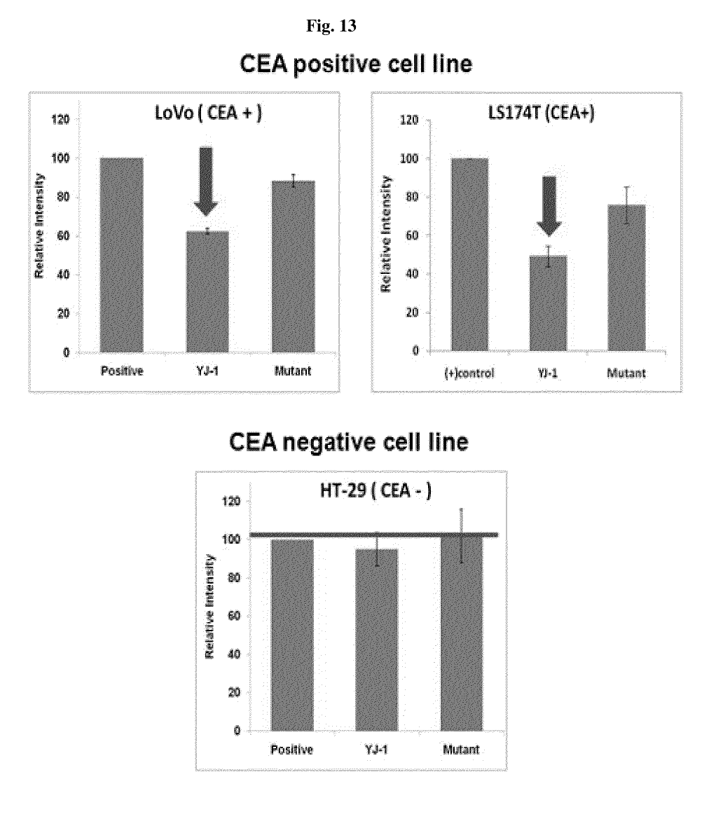

The result is shown in FIG. 13. As shown in FIG. 13, it is confirmed that in CEA-positive cells, i.e., LS174T and LoVo cells, CEA aptamer (YJ-1) specifically inhibits migration of cancer cells, while in CEA-negative cell, i.e., HT29 cell, CEA aptamer (YJ-1) does not have influence on the migration of cancer cell.

13. Inhibition of CEA Dependent Anoikis Resistancy by RNA Aptamer

To confirm whether RNA aptamer (YJ-1) inhibits the function of CEA for inhibiting Anoikis in the steps of metastasis, Anoikis inducing Assay was performed. Caspase 8 Colorimetric Assay Kit (Millipore) was used. One day before, a polyHEMA coated plate was manufactured (polyHEMA was loaded on a plate at a concentration of 3 mg/cm², and O/N incubated in a clean bench). And, selected RNA aptamer (for example, YJ-1 aptamer) or its mutant aptamer (for example, Mutant YJ-1 aptamer) was transfected into LoVo cell twice (TransIT-TKO® (Minis Bio LLC); 50 nM aptamer 16 hrs), and then, the transfected 2×10⁶ cells were loaded on the previously manufactured polyHEMA coated plate, and treated with selected RNA aptamer (for example, YJ-1 aptamer: 40 μg/ml) or its mutant aptamer (for example, Mutant YJ-1 aptamer: 40 μg/ml) in a 37° C. CO₂ incubator and suspension incubated for 24 hours. And then, the cells were treated with 100 μL of 1× Cell Lysis Buffer to obtain total protein, which were then treated with Caspase 8 substrate and reacted for 1-2 hours, and measured at 410 nm (microplate reader 550, Biorad).

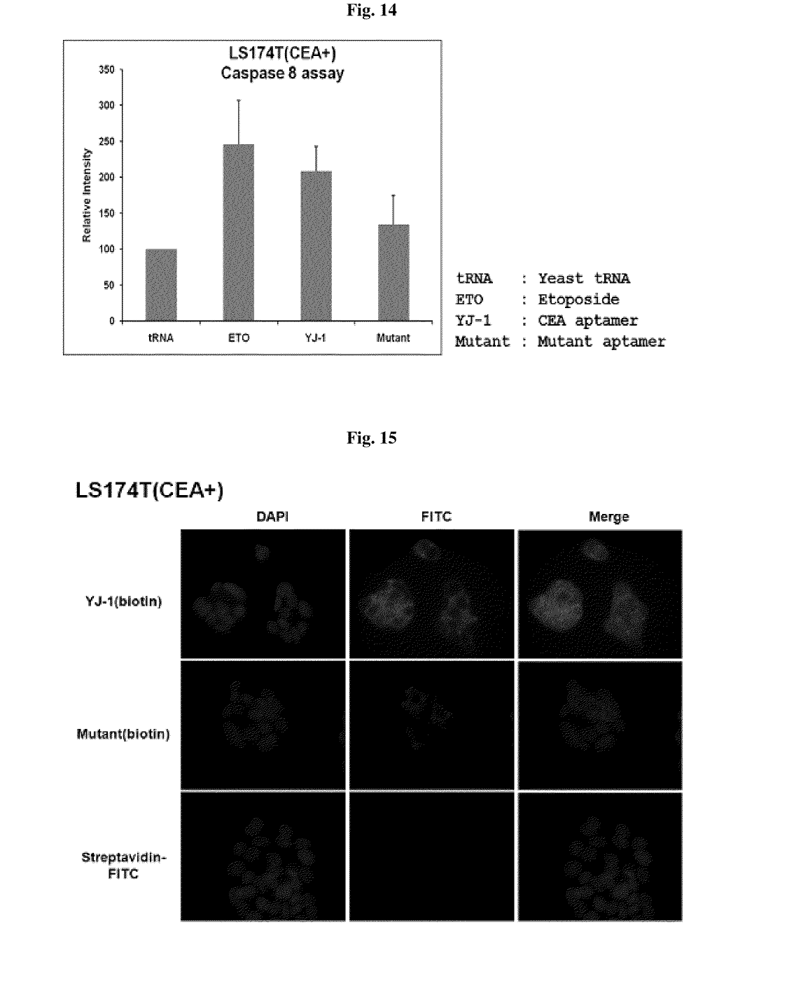

The result is shown in FIG. 14. As shown in FIG. 14, it is confirmed that in CEA-positive cell, i.e., LoVo cell, CEA aptamer (YJ-1) specifically induces Anoikis of cancer cell similarly to positive control Etoposide (sigma) treatment, while mutant aptamer does not have influence on inducing of cancer cell Anoikis.

14. Fluorescence Staining of CEA Positive Cell by RNA Aptamer

To confirm specific binding to CEA-positive cell using selected RNA aptamer, Fluorescence aptamer cell staining assay was performed. One day before, a cover slip was fixed to a 100 mm dish using 0.1% Gelatin. Next day, the number of incubated cells were determined with Hemocytometer (1×10⁶ cells/10 ml), and then, they were seeded into the cover slip-fixed 100 mm dish. After incubating in a 37° C. 5% CO2 incubator for 24 hours, the cover slip was moved to a 12 well plate and fixed with 4% paraform aldehyde solution (sigma). And then, the cells were treated with primary Blocking buffer (TNB buffer) at room temperature for 1 hour, and then treated with secondary Blocking buffer (PBS-MgC12 with tRNA (10 ug/ul), poly IC (1 ug/ul)) for 40 minutes. After washing with PBS Mg buffer (PBS with 1.5 mM MgC12) once, they were treated with selected Biotin-tagged RNA aptamer (for example, YJ-1 aptamer 500 nM) or its mutant aptamer (for example, Mutant YJ-1 aptamer 500 nM) at room temperature for 30 minutes. After washing with PBS Mg buffer 3 times, they were treated with fluorescence-tagged Streptavidin (BD bioscience.) at a concentration of 1:100 at room temperature for 1 hour. After washing with PBS-T Mg buffer (PBS with 1.5 mM MgCl₂, 0.05% tween20) 3 times, they were mounted with a mounting solution (Slowfade Gold antifade reagent with DAPI, invitrogen), and observed by fluorescent microscope (Carl Zeiss, Inc.).

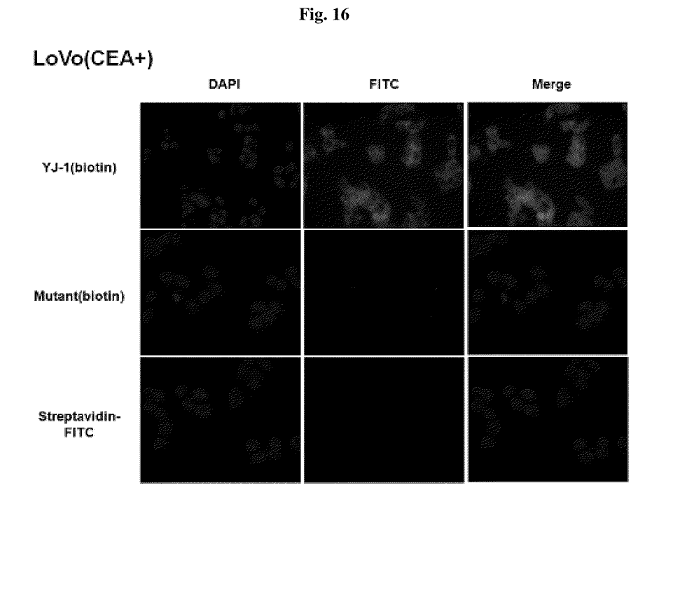

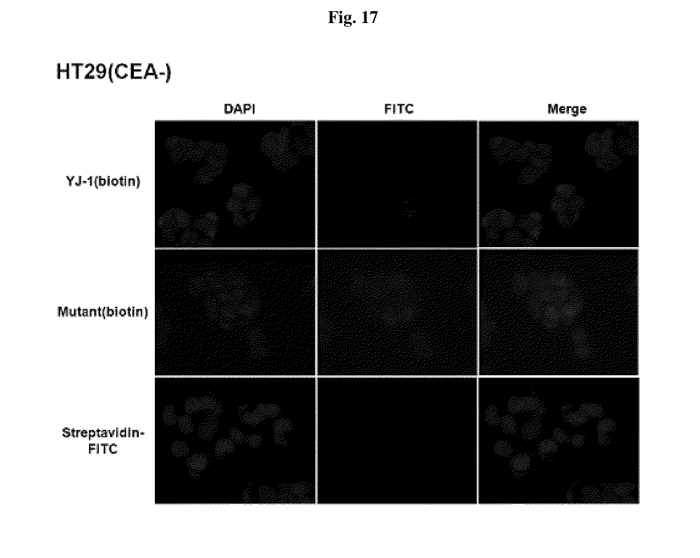

The results are shown in FIG. 15, FIG. 16, and FIG. 17, respectively. It is confirmed through flurescence staining that CEA aptamer (YJ-1) specifically binds to CEA-positive cancer cells, i.e., LS174T and LoVo cells (FIG. 15, FIG. 16). And, it is confirmed that CEA aptamer (YJ-1) does not bind to CEA-negative cell, i.e., HT29 cell (FIG. 17). It is also confirmed that control mutant aptamer fails to bind to the surface of cancer cell irrespectively of CEA expression (FIG. 15, FIG. 16, FIG. 17).

In conclusion, RNA aptamers of SEQ ID NO: 1 to SEQ ID NO: 14 including optimized YJ-1 RNA aptamer specifically bind to N domain of CEA, and thus, inhibits aggregation of CEA-positive cancer cells, inhibits ECM adhesion of some CEA-positive cancer cells, and inhibits specifically to CEA-positive cells in vitro invasion or in vitro migration, which is an important process of metastasis. And, it is confirmed that Anoikis resistance induced by CEA that is characteristic of metastatic cancer cell is aptamer specifically inhibited, and the RNA aptamer effectively inhibits liver metastasis in an animal model of liver metastasis of colon cancer, indicating that it may be useful for inhibiting liver metastasis induced by CEA. Finally, it is confirmed by fluorosecence staining of CEA-positive cells using RNA aptamer that CEA aptamer (YJ-1) specifically binds to CEA-positive cancer cell, indicating that it may be useful for specific diagnosis of CEA-overexpressing cancer cell.

Accordingly, the metastasis inhibitor according to the present invention may effectively inhibit metastasis to other tissues, one of important problems of cancer treatment, and thus, may be used as a therapeutic agent for inhibiting metastasis, and may be used as a cancer therapeutic agent through combined administration with other anticancer agents, and the RNA aptamers may be used for a diagnosis agent capable of measuring and expecting the degree of metastasis to other tissues of colon cancer, etc.

1. RNA aptamer specifically binding to a linkage region between N domain and A1 domain of carcinoembryonic antigen (CEA), comprising continuous 35 or more bases comprising the nucleotide sequence from 9^{th} to 43^{rd} positions of following SEQ ID NO: 13:
<SEQ ID NO: 13>
GCGGAAGCGUGCUGGGCUAGAAUAAUAAUAAGAAAACCAGUA
CUUUCGU.

2. The RNA aptamer according to claim 1, wherein the RNA aptamer comprises the nucleotide sequence of SEQ ID NO: 13 or 14.

3. The RNA aptamer according to claim 1, wherein the RNA aptamer is modified by at least one method selected from:
using C(cytosine) and U(uracil) wherein 2′ hydroxyl group is substituted by fluoro group; and
attaching cholesterol at 5′ end, and attaching idT (inverted deoxy thymidylate) at 3′ end.

4. A composition for preventing or inhibiting cancer metastasis containing the RNA aptamer of claim 1 as an active ingredient.

5. The composition according to claim 4, wherein the RNA aptamer comprises the nucleotide sequence of SEQ ID NO: 13 or 14.

6. The composition according to claim 4, wherein the cancer is selected from the group consisting of colon cancer, stomach cancer, pancreatic cancer, and lung cancer.

7. The composition according to claim 4, wherein the cancer metastasis is a cancer metastasis to liver.

8. A method for preventing or inhibiting cancer metastasis, comprising administering the RNA aptamer of claim 1 to a patient in need of inhibition of metastasis.

9. The method according to claim 8, wherein the RNA aptamer comprises the nucleotide sequence of SEQ ID NO: 13 or 14.

10. The method according to claim 9, wherein the cancer is selected from the group consisting of colon cancer, stomach cancer, pancreatic cancer, and lung cancer.

11. The method according to claim 8, wherein the cancer metastasis is a cancer metastasis to liver.

12. A composition for diagnosis of cancer metastasis containing the RNA aptamer of claim 1.

13. The composition for diagnosis of cancer metastasis according to claim 12, wherein the RNA aptamer comprises the nucleotide sequence of SEQ ID NO: 13 or 14.

14. The composition for diagnosis of cancer metastasis according to claim 12, wherein the cancer is selected from the group consisting of colon cancer, stomach cancer, pancreatic cancer, and lung cancer.

15. The composition for diagnosis of cancer metastasis according to 12, wherein the cancer metastasis is a cancer metastasis to liver.

16. A method of diagnosis of cancer metastasis comprising:
treating a sample with the RNA aptamer of claim 1, and
detecting binding of the RNA aptamer and a linkage region between N domain and A1 domain of CEA,
wherein it is determined that cancer metastasis occurs when the binding is detected.

17. The method of diagnosis of cancer metastasis according to 16, wherein the RNA aptamer comprises the nucleotide sequence of SEQ ID NO: 13 or 14.

18. The method of diagnosis of cancer metastasis according to 16, wherein the cancer is selected from the group consisting of colon cancer, stomach cancer, pancreatic cancer, and lung cancer.

19. The method of diagnosis of cancer metastasis according to 16, wherein the cancer metastasis is a cancer metastasis to liver.

20. A method of imaging CEA-expressing cancer cells, which comprises:
applying the RNA aptamer of claim 1, which is labeled with a fluorescence or radioisotope, to a living body or an isolated tissue or cell; and
detecting the fluorescence or radioisotope.
